(19) United States
(12) Patent Application Publication
(10) Pub. No.: US 2024/0409951 A1
(43) Pub. Date: Dec. 12, 2024

(54) SYMRK PHOSPHORYLATION FOR ROOT NODULE ORGANOGENESIS

(71) Applicant: Aarhus Universitet, Aarhus (DK)

(72) Inventors: Kasper Røjkjær ANDERSEN, Aarhus (DK); Nikolaj Birkebaek ABEL, Skanderborg (DK); Malita Malou Malekzadeh NØRGAARD, Harlev J (DK); Jens Stougaard JENSEN, Aarhus (DK); Kira Dorothea GYSEL, Aarhus (DK); Simon Boje HANSEN, Aarhus (DK)

(73) Assignee: Aarhus Universitet, Aarhus (DK)

(21) Appl. No.: 18/737,606

(22) Filed: Jun. 7, 2024

Related U.S. Application Data
(60) Provisional application No. 63/507,414, filed on Jun. 9, 2023.

(51) Int. Cl. C12N 15/82; C12N 9/12; C12N 9/22; C12N 15/11

(52) U.S. Cl. CPC C12N 15/8262; C12N 9/12; C12N 9/22; C12N 15/111; C12N 15/8201; C12N 2310/20; C12Y 207/11001

(57) ABSTRACT

The present disclosure relates to modified plant SYMRK polypeptides that constitutively induces symbiotic organogenesis or induces symbiotic organogenesis in the absence of rhizobial bacteria and/or arbuscular mycorrhizal fungi recognized by the plant at a higher level than the unmodified plant SYMRK polypeptide under the same conditions and use thereof in plants.

### CROSS-REFERENCE TO RELATED APPLICATIONS

This application claims the benefit of U.S. Provisional Application No. 63/507,414, filed Jun. 9, 2023, which is hereby incorporated by reference in its entirety.

### REFERENCE TO AN ELECTRONIC SEQUENCE LISTING

The content of the electronic sequence listing (794542002600seqlist.xml; Size: 49,050 bytes; and Date of Creation: Jun. 6, 2024) is herein incorporated by reference in its entirety.

### TECHNICAL FIELD

The present disclosure relates to modified plant SYMRK polypeptides that constitutively induce symbiotic organogenesis or induce symbiotic organogenesis in the absence of rhizobial bacteria and/or arbuscular mycorrhizal fungi recognized by the plant at a higher level than the unmodified plant SYMRK polypeptide under the same conditions and use thereof in plants.

### BACKGROUND

Legumes can overcome nitrogen limitations in soil by acquiring atmospheric nitrogen through symbiosis with nitrogen fixing rhizobia bacteria (Oldroyd, Nat Rev Microbiol (2013) 11:252-263). This symbiosis comes at a high energy cost for the plant and an increased risk of pathogen infection. Therefore, tight regulation of the symbiotic process is important, both to limit the number of symbiotic events and to ensure recognition of symbiotic bacteria (Roy et. al., Plant Cell (2020) 32:15-41). Extracellular Nod factor perception is done by Nod factor receptor kinases Nod factor receptor 1 (NFR1) and Nod factor receptor 5 (NFR5) (Radutoiu et al., Nature (2003) 425 (6958): 585-92, Madsen et al., Nature (2003) 435 (6958): 637-40), and the Malectin-like/leucine-rich repeat receptor kinase Symbiosis receptor-like kinase (SYMRK) (Stracke et al., Nature (2002) 417 (6892): 959-62), which perceive the Nod factors and initiate the signaling process that results in the formation of symbiotic infection threads and initiation of nodule organogenesis. Together, NFR1 and NFR5 make up the core complex for root nodule symbiosis (RNS) signaling, and SYMRK acts downstream and is required for RNS and mycorrhization. Both NFR1 and SYMRK are active kinases (Radutoiu et al., Nature (2003) 425 (6958): 585-92, Yoshida et al. Journal of Biological Chemistry (2004) 280:9203-9209), whereas NFR5 is a pseudokinase (Madsen et al., Nature Communications, (2010) 1 (10); Madsen et al., Plant J. (2011) 65 (3) 404-417).

Successful establishment of RNS is dependent on a functional SYMRK (Stracke 2002), and even spontaneous nodule formation through forced interaction between NFR1 and NFR5 has been shown to be fully dependent on the presence of SYMRK (Rübsam et al., Science (2023) 379 (6629): 272-277)). SYMRK is a key component of the common symbiotic pathway (CSP) driving symbiosis with rhizobia, Frankia and Arbuscular mycorrhiza (AM) (Huisman and Geurts, Plants Commun (2020) 1). It is not known how the signal is transmitted from the Nod factor receptors to SYMRK and further down the CSP, but it is speculated that receptor-interacting proteins and secondary messengers are involved (Jhu and Oldroyd, PLOS Biol (2023) 21: e30001982). One receptor-interacting protein has been identified as being a cytosolic kinase, NiCK4, which is phosphorylated in the presence of NFR1 in vitro (Wong et al., Proc Natl Acad Sci USA (2019) 116:14339-14348). The NiCK4 protein seems not to have any influence on SYMRK, as no interaction or transphosphorylation between the two proteins were observed (Wong et al., Proc Natl Acad Sci USA (2019) 116:14339-14348).

Phosphorylation of serine and threonine are among the common regulatory mechanisms for receptor kinases in both plant and animal cells. Protein phosphorylation signaling has been shown to control pathways in all aspects of plant life, including defense responses to pathogens and development (Hohmann et al., Annu Rev. Plant Biol (2017) 68:109-137). As a prime example of the importance of phosphorylation, the multi-functional receptor kinase BAK1 is involved in both brassinosteroid signaling by interaction with BRI1 (Nam et al., Cell (2002) 110 (2) 203-12) and defense responses when interacting with FLS2 in a ligand-dependent manner (Chinchilla et al., Nature (2007) 448:997-500). It has been shown that conserved phosphorylation sites in the C-terminal tail of BAK1 are required for the role in immunity but not for the role in brassinosteroid signaling (Parraki, Nature (2018) 561:248-252).

Root nodule symbiosis is regulated by phosphorylation signaling. It has been shown that correct phosphorylation at the activation loops of Lotus japonicus (Lotus) NFR1 and of its homologue in Medicago truncatula (Medicago), LYK3, is required for nodule formation (Madsen et al., Nature Communications, (2010) 1 (10); Madsen et al., Plant J. (2011) 65 (3) 404-417; Klaus-Heisen et al., Journal of Biological Chemistry (2011) 286:11202-11210). Further downstream in the CSP, phosphorylation of CYCLOPS and CCAMK is also essential for nodule development (Singh et al., Cell Host Microbe (2014) 15:139-152, Tirichine et al., Nature (2006) 441:1153-1156). SYMRK in Arachis hypogaea has been shown to be regulated by phosphorylation (Saha et al, Plant Physiol (2016) 171:71-81) and the activity of Lotus SYMRK kinase activity is regulated by phosphorylation at T760 (Yoshida et al., Journal of Biological Chemistry (2005) 280:9203-9209).

There exists a need to identify the specific phosphorylation sites in SYMRK involved in symbiosis signaling in nodulating legume species. The identification of these phosphorylation sites will allow the engineering of existing SYMRK proteins in non-legume species. The engineering of SYMRK, an essential component of the CSP, is a vital step in engineering nodulation in non-legume species.

### BRIEF SUMMARY

The present disclosure identifies phosphorylation sites in SYMRK that are essential for successful symbiosis but are also sufficient to drive organogenesis in the absence of symbiotic bacteria. In particular, four serine residues in the C-terminal tail of SYMRK were identified that were sufficient to drive nodule organogenesis. A phospho-mimic of these four serine residues resulted in the formation of spontaneous nodules in the absence of symbiotic bacteria. It was also surprisingly discovered that substituting non-phosphorylatable amino acid residues in the kinase core can activate SYMRK.

An aspect of the disclosure includes a modified plant SYMRK polypeptide including (i) substitution of one or more, two or more, three or more, or all four phosphorylatable amino acid residues with a phosphomimetic amino acid residue, wherein the one or more, two or more, three or more, or all four phosphorylatable amino acid residues correspond to amino acids S877, S885, S889, or S893 of SEQ ID NO: 2, and/or (ii) substitution of one or more, two or more, three or more, four or more, or all five phosphorylatable amino acid residues with a non-phosphomimetic amino acid residue, wherein the one or more, two or more, three or more, or all four phosphorylatable amino acid residues correspond to amino acids S724, S731, S742, S751, or S754 of SEQ ID NO: 2. In a further embodiment of this aspect, the phosphomimetic amino acid residue is aspartic acid or glutamic acid, preferably aspartic acid. In an additional embodiment of this aspect, which may be combined with any of the preceding embodiments, the non-phosphorylatable amino acid residue is alanine or glycine, preferably alanine. In still another embodiment of this aspect, which may be combined with any of the preceding embodiments, the phosphorylatable amino acid residues are serine, tyrosine, or threonine. In a further embodiment of this aspect, which may be combined with any of the preceding embodiments the plant SYMRK polypeptide includes a polypeptide with at least 70% identity, at least 80% identity, at least 90% identity, at least 95% identity, or at least 99% identity to a protein selected from the group of SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8 or a functional fragment or conserved domain thereof. In yet another embodiment of this aspect, which may be combined with any of the preceding embodiments, the modified plant SYMRK polypeptide constitutively induces symbiotic organogenesis or induces symbiotic organogenesis in the absence of rhizobial bacteria and/or arbuscular mycorrhizal fungi recognized by the plant at a higher level than the unmodified plant SYMRK polypeptide under the same conditions. In a further embodiment of this aspect, the organogenesis is nodule formation, arbuscule or vesicle formation, or lateral root formation, preferably nodule formation. In yet another embodiment of this aspect, which may be combined with any preceding embodiment that has the modified plant SYMRK polypeptide constitutively inducing symbiotic organogenesis, the symbiosis is nitrogen-fixing nodulation symbiosis or arbuscular mycorrhizal symbiosis, preferably nitrogen-fixing nodulation symbiosis. In a further embodiment, the modified plant SYMRK polypeptide comprises an active kinase domain.

Some aspects of the disclosure relate to a genetically modified plant or part thereof including the modified plant SYMRK receptor polypeptide of any one of the preceding embodiments. In a further embodiment of this aspect, the modified plant SYMRK polypeptide constitutively induces symbiotic organogenesis or induces symbiotic organogenesis in the absence of rhizobial bacteria and/or arbuscular mycorrhizal fungi recognized by the plant at a higher level than the unmodified plant SYMRK polypeptide under the same conditions. In still another embodiment of this aspect, which may be combined with any of the preceding embodiments, the organogenesis is nodule formation, arbuscule or vesicle formation, or lateral root formation, preferably nodule formation. In yet another embodiment of this aspect, which may be combined with any of the preceding embodiments, the symbiosis is nitrogen-fixing nodulation symbiosis or arbuscular mycorrhizal symbiosis, preferably nitrogen-fixing nodulation symbioses. In a further embodiment, the modified plant SYMRK polypeptide comprises an active kinase domain. An additional embodiment of this aspect, which may be combined with any of the preceding embodiments, further includes a genetically modified plant NFR1 LysM receptor polypeptide and/or a genetically modified plant NFR5 LysM receptor polypeptide.

Additional aspects of the disclosure relate to methods of producing the genetically modified plant or part thereof of any of the preceding embodiments, including introducing a genetic alteration to the plant including a first nucleic acid sequence encoding the modified plant SYMRK receptor polypeptide. In a further embodiment of this aspect, the nucleic acid sequence is operably linked to a promoter, wherein the promoter is a root active promoter, an inducible promoter, a constitutive promoter, or a combination thereof. In still another embodiment of this aspect, the promoter is a root active promoter, and wherein the promoter is selected from the group of a NFR1 promoter, a NFR5 promoter, a LYK3 promoter, a CERK6 promoter, a NFP promoter, a Lotus japonicus NFR5 promoter (SEQ ID NO: 9), a Lotus japonicus NFR1 promoter (SEQ ID NO: 10), a Lotus japonicus CERK6 promoter (SEQ ID NO: 11), a Medicago truncatula NFP promoter (SEQ ID NO: 12), a Medicago truncatula LYK3 promoter (SEQ ID NO: 13), a maize allothioneine promoter, a chitinase promoter, a maize ZRP2 promoter, a tomato LeExtl promoter, a glutamine synthetase soybean root promoter, a RCC3 promoter, a rice antiquitine promoter, a LRR receptor kinase promoter, or an Arabidopsis pCO2 promoter. In a further embodiment of this aspect, the promoter is a constitutive promoter, and wherein the promoter is selected from the group of a CaMV35S promoter, a derivative of the CaMV35S promoter, a maize ubiquitin promoter, a trefoil promoter, a vein mosaic cassava virus promoter, or an Arabidopsis UBQ10 promoter. In yet another embodiment of this aspect, the nucleic acid sequence is inserted into the genome of the plant so that the nucleic acid sequence is operably linked to an endogenous promoter, and wherein the endogenous promoter is a root active promoter. An additional embodiment of this aspect, which may be combined with any of the preceding embodiments, further includes introducing one or more genetic alterations to the plant comprising a second nucleic acid sequence encoding a modified plant NFR1 LysM receptor polypeptide and/or a third nucleic acid sequence encoding a modified plant NFR5 LysM receptor polypeptide.

Further aspects of the disclosure relate to methods of producing the genetically modified plant or part thereof of any of the preceding embodiments, including genetically modifying the plant or part thereof by transforming the plant or part thereof with one or more gene editing components that target an endogenous nuclear genome sequence encoding an endogenous plant SYMRK receptor polypeptide and introduce (i) the substitution of one or more, two or more, three or more, or all four phosphorylatable amino acid residues with a phosphorylatable amino acid residue, wherein the one or more, two or more, three or more, four or more, or all five phosphorylatable amino acid residues correspond to amino acids S877, S885, S889, or S893 of SEQ ID NO: 2, and/or (ii) the substitution of one or more, two or more, three or more, four or more, or all five phosphorylatable amino acid residues with a non-phosphomimetic amino acid residue, wherein the one or more, two or more, three or more, or all four phosphorylatable amino acid residues correspond to amino acids S724, S731, S742, S751, or S754 of SEQ ID NO: 2. In an additional embodiment of this aspect, the one or more gene editing components include a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence. Yet another embodiment of this aspect, which may be combined with any of the preceding embodiments, further includes genetically modifying the plant or part thereof by transforming the plant or part thereof with one or more gene editing components that target a second endogenous nuclear genome sequence encoding an endogenous plant NFR1 LysM receptor polypeptide for modification and/or that target a third endogenous nuclear genome sequence encoding an endogenous plant NFR5 LysM receptor polypeptide for modification.

Still further aspects of the disclosure relate to an expression vector, isolated DNA molecule, or recombinant nucleic acid including a nucleic acid sequence encoding the modified plant SYMRK receptor polypeptide of any one of the preceding embodiments, optionally operably linked to at least one expression control sequence. In further embodiments of this aspect, the at least one expression control sequence includes a promoter selected from the group of a root active promoter, a constitutive promoter, and a combination thereof. In additional embodiments of this aspect, the promoter is a root active promoter, and wherein the promoter is selected from the group of a NFR1 promoter, a Lotus japonicus NFR5 promoter (SEQ ID NO: 9), a Lotus japonicus NFR1 promoter (SEQ ID NO: 10), a Lotus japonicus CERK6 promoter (SEQ ID NO: 11), a Medicago truncatula NFP promoter (SEQ ID NO: 12), a Medicago truncatula LYK3 promoter (SEQ ID NO: 13), a maize allothioneine promoter, a chitinase promoter, a maize ZRP2 promoter, a tomato LeExt1 promoter, a glutamine synthetase soybean root promoter, a RCC3 promoter, a rice antiquitinc promoter, a LRR receptor kinase promoter, or an Arabidopsis pCO2 promoter. In still further embodiments of this aspect, the promoter is constitutive promoter, and wherein the promoter is selected from the group of a CaMV35S promoter, a derivative of the CaMV35S promoter, a maize ubiquitin promoter, a trefoil promoter, a vein mosaic cassava virus promoter, or an Arabidopsis UBQ10 promoter.

Yet further aspects of the disclosure relate to a bacterial cell or an Agrobacterium cell including the expression vector, isolated DNA molecule, or recombinant nucleic acid of any one of the preceding embodiments.

Additional aspects of the disclosure relate to a genetically modified plant, plant part, plant cell, or seed including the expression vector, isolated DNA molecule, or recombinant nucleic acid of any one of the preceding embodiments.

Further aspects of the disclosure relate to a composition or kit including the expression vector, isolated DNA molecule, or recombinant nucleic acid of any one of the preceding embodiments, the bacterial cell or Agrobacterium cell of the preceding embodiments, or the genetically modified plant, plant part, plant cell, or seed of the preceding embodiments.

Yet further aspects of the disclosure relate to a non-regenerable part or cell of the genetically modified plant or part thereof of any one of the preceding embodiments.

Yet another aspect of the disclosure relates to methods of constitutively inducing symbiotic organogenesis or inducing symbiotic organogenesis in the absence of rhizobial bacteria and/or arbuscular mycorrhizal fungi recognized by the plant including introducing a genetic alteration via the expression vector, isolated DNA molecule, or recombinant nucleic acid of any one of the preceding embodiments.

Yet another aspect of the disclosure relates to increasing symbiotic function of a SYMRK by (i) substituting one or more, two or more, three or more, or four phosphorylatable amino acid residues with a phosphomimetic amino acid residue, wherein the one or more, two or more, three or more, or four phosphorylatable amino acid residues correspond to amino acids S877, S885, S889, or S893 when aligned to SEQ ID NO: 2, and/or (ii) substituting one or more, two or more, three or more, four or more, or five phosphorylatable amino acid residues with a non-phosphorylatable amino acid residue, wherein the one or more, two or more, three or more, four or more, or five phosphorylatable amino acid residues correspond to amino acids S724, S731, S742, S751, or S754 when aligned to SEQ ID NO: 2, resulting in a modified SYMRK. In some embodiments the increase in symbiotic function is measured in comparison to the WT SYMRK in the absence of rhizobial bacteria and/or arbuscular mycorrhizal fungi.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

FIGS. 1A-1E show crystal structures and phosphorylatable amino acid residue data of SYMRK. FIG. 1A and FIG. 1B show two images depicting the three-dimensional crystal structure of LjSYMRK. FIG. 1A is colored and labeled to emphasize secondary structure, with the catalytic loop in yellow (and identified by the HRD-motif), the activation loop in green (and identified by the DFG-motif), alpha helices in gray (and labeled αA-αI), and beta sheets in dark blue (and labeled β0-β5), and particular regions labeled alongside. FIG. 1B is colored and labeled to highlight the important amino acids found in the structure of SYMRK, and the ones known for the regulatory and catalytic spine, with the catalytic spine highlighted in yellow (V608, A620, I726, L727, L728, L675, V786, and 1790), the regulatory spine highlighted in red (L653, L642, F739, and H718), and the gatekeeper residue highlighted in green (Y667) labeled. Individual residues are labeled alongside. FIG. 1C shows a table of results from a mass spectrometric approach to identify phosphorylatable amino acid residues (Phosphorsite) on SYMRK in vitro, showing that serine and threonine residues were identified as phosphorylated on LjSYMRK. FIG. 1D shows the phosphorylatable amino acid residues from FIG. 1C visualized on the structure from FIG. 1A and FIG. 1B, with the predicted C-terminal tail (not determined as a crystal structure). Residues S724, S731, S742, S751, and S754 are in the kinase core, of which site 724 is in the catalytic loop, site 731 is in the Mg binding loop, and sites 742, 751, and 754 are in the activation loop. Residues 877 through 918 are in the C-terminal tail. FIG. 1E shows the construct design for testing phospho-mimics (D) and phospho-ablation (A) in planta, with phospho-ablation constructs mutating serine/threonine to alanine, and phospho-mimetic constructs mutating serine/threonine to aspartic acid (top). The structural similarities between the chemical structures of aspartic acid and phosphor-serine are shown at the bottom.

FIGS. 2A-2D show that phosphorylatable amino acid residues in the C-terminal tail are required for root nodule symbiosis. FIG. 2A shows an alignment of SYMRK mutants used, with residues that match at a given position shaded in the same color. From top to bottom, these are SEQ ID NO: 14, SEQ ID NO: 15, SEQ ID NO: 16, SEQ ID NO: 17, SEQ ID NO: 18, SEQ ID NO: 19, SEQ ID NO: 20, and SEQ ID NO: 21. FIG. 2B shows a plot displaying the number of pink nodules per plant as measured by the number of pink (infected) nodules on hairy roots expressing indicated SYMRK mutants in symrk-3 plants under the native promoter. Data were collected four weeks after inoculation with rhizobia. FIG. 2C shows a plot displaying results of NanoDSF thermal stability assays of SYMRK kinase domains purified from E. coli and supplemented with 5 mM MgCl2 and 1 mM ATP. Thermal stabilization upon incubation with Mg and ATP indicates ATP binding for SYMRK WT, 4A, and 4D, but not K622E. FIG. 2D shows blots containing results of [γ-32P] auto- and trans-phosphorylation assays of SYMRK kinase domains incubated with Myelin basic protein (MBP). Phosphorylated SYMRK and MBP were separated by SDS-PAGE and visualized by Coomassic blue staining (left) and autoradiography (right).

FIGS. 3A-3D show that phosphor mimics in the C-terminal tail are sufficient to drive organogenesis. FIG. 3A shows a plot displaying the number of pink (infected) and white (uninfected) nodules on hairy roots expressing indicated SYMRK mutants in symrk-3 under the native promoter in the presence of rhizobia. Data were collected four weeks after inoculation with rhizobia. FIG. 3B shows a plot displaying the number of white (uninfected) nodules on hairy roots expressing indicated SYMRK mutants in symrk-3 plants under the native promoter in the absence of rhizobia. Data were collected four weeks after hairy root formation. FIG. 3C shows a plot displaying the number of white (uninfected) nodules on hairy roots expressing indicated SYMRK mutants in the indicated mutant backgrounds under the native promoter in the absence of rhizobia. Data were collected four weeks after hairy root formation. FIG. 3D shows representative microscopy images of roots from FIG. 3C, with bright field images shown along the top, and YFP fluorescent images shown along the bottom, in which nuclear-localized YFP was used as a transformation marker.

FIGS. 4A-4F show SDS-PAGE gels and size exclusion chromatography (SEC) plots on the purified kinase domains used for activity tests and ATP binding assays. In the SEC plots, the y-axes represent milli absorbance units (mAU). In FIGS. 4A-4D and FIG. 4F, the SEC plot is displayed on the left, and the SDS-PAGE gel is shown on the right. After the SEC run the proteins were collected in fractions (indicated with numbers) and run on the SDS-PAGE gel to confirm the size of the protein. Pooled fractions are indicated with dashed lines in chromatograms and horizontal black lines above fraction lanes in SDS-PAGE gels. FIG. 4A shows results from SYMRK WT proteins. FIG. 4B shows results from SYMRK K622E proteins. FIG. 4C shows results from SYMRK 4A proteins. FIG. 4D shows results from SYMRK 4D proteins. FIG. 4E shows comparative analytical SEC results from SYMRK WT (black), K622E (blue), 4A (green), and 4D (red) proteins. FIG. 4F shows results from NFR1 WT proteins.

FIG. 5 shows an amino acid alignment of the C-terminal tail of SYMRK protein sequences from Lotus japonicus (Lj; full length sequence=SEQ ID NO: 2; UniProt ID Q8LKX1; alignment sequence=SEQ ID NO: 22), Medicago truncatula (Mt; full length sequence=SEQ ID NO: 4; UniProt ID Q8L4H4; alignment sequence=SEQ ID NO: 23), Manihot esculenta (Me; full length sequence=SEQ ID NO: 5; UniProt ID A0A2C9UVV4; alignment sequence=SEQ ID NO: 24), Solanum lycopersicum (Sl; full length sequence=SEQ ID NO: 3; UniProt ID Q2TDW9; alignment sequence=SEQ ID NO: 25), Oryza sativa (Os; full length sequence=SEQ ID NO: 6; UniProt ID Q7F110; alignment sequence=SEQ ID NO: 26), Zea mays (Zm; full length sequence=SEQ ID NO: 1; UniProt ID Q208N5; alignment sequence=SEQ ID NO: 27), Hordeum vulgare (Hv; full length sequence=SEQ ID NO: 7; UniProt ID A0A816XF58; alignment sequence=SEQ ID NO: 28) and Arabidopsis thaliana (At; full length sequence=SEQ ID NO: 8; UniProt ID COLGI2; alignment sequence=SEQ ID NO: 29) and consensus sequence (SEQ ID NO: 30). Phosphorylatable amino acid residues identified in this study are indicated at the top.

FIG. 6 shows fluorescent microscopy images of Nicotiana benthamiana leaves expressing the SYMRK mutant indicated above each image tagged with mCherry.

FIG. 7 shows microscopy images of representative roots and nodules on hairy roots expressing the SYMRK mutants indicated above the images in symrk-3 plants under the native promoter, four weeks after inoculation with rhizobia. Top: bright field images; middle: YFP fluorescence channel displaying nuclear-localized YFP, which was used as a transformation marker; bottom: DsRED fluorescence channel displaying DsRED labelled rhizobia.

FIGS. 8A-8C show protein data indicating that five phosphorylatable amino acid residues in the activation zone negatively regulate SYMRK kinase activity and symbiotic function. FIG. 8A shows an amino acid alignment of WT-SYMRK (SEQ ID NO: 31), 5A-SYMRK (SEQ ID NO: 32), and 5D-SYMRK (SEQ ID NO: 33), including a consensus sequence (SEQ ID NO: 34). FIG. 8B shows a plot displaying the number of nodules per plant on hairy roots expressing the indicated SYMRK mutants in symrk-3 plants under the native promoter in the presence of rhizobia. Data were collected four weeks after inoculation with rhizobia. FIG. 8C shows a blot of results of [γ-32P] auto- and trans-phosphorylation assays of the LjSYMRKk (LjSYMRK kinase) 5D and 5A mutants compared to WT and K622A LjSYMRKk incubated with Myelin basic protein (MBP). Phosphorylated LjSYMRKk and MBP were separated by SDS-PAGE and visualized by Coomassie blue staining (top) and autoradiography (bottom).

FIGS. 9A-9B show nodulation data indicating that spontaneous organogenesis is dependent on kinase activity. FIG. 9A shows a crystal structure of SYMRK 4D, a phosphor mimic mutant (substituting S with D) version of SYMRK at S877, S885, S889, and S893. FIG. 9B shows a plot displaying the number of white (uninfected) nodules on hairy roots expressing indicated SYMRK mutants (4D and 4D with a 622E mutation) in symrk-3 plants under the native promoter in the absence of rhizobia.

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

### Modified Plant SYMRK Receptor Polypeptides and Related Methods

An aspect of the disclosure includes a modified plant SYMRK polypeptide including (i) substitution of one or more, two or more, three or more, or all four phosphorylatable amino acid residues corresponding to amino acids S877, S885, S889, and S893 of SEQ ID NO: 2 (LjSYMRK UniProt ID Q8LKX1) with a phosphomimetic amino acid residue, and/or (ii) substitution of one or more, two or more, three or more, four or more, or all five phosphorylatable amino acid residues corresponding to amino acids S724, S731, S742, S751, and S754 of SEQ ID NO: 2 (LjSYMRK UniProt ID Q8LKX1) with a non-phosphorylatable amino acid residue. In a further embodiment of this aspect, the phosphomimetic amino acid residue is aspartic acid or glutamic acid, preferably aspartic acid. In an additional embodiment of this aspect, which may be combined with any of the preceding embodiments, the non-phosphorylatable amino acid residue is alanine or glycine, preferably alanine. In still another embodiment of this aspect, which may be combined with any of the preceding embodiments, the phosphorylatable amino acid residues are serine, tyrosine, and/or threonine. In a further embodiment of this aspect, which may be combined with any of the preceding embodiments the plant SYMRK polypeptide includes a polypeptide with at least 70% identity, at least 80% identity, at least 90% identity, at least 95% identity, or at least 99% identity to a protein selected from the group of SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8 or a functional fragment or conserved domain thereof. In a further embodiment of this aspect, substitution of one or two phosphorylatable amino acid residues in step (i) is sufficient. The phosphorylatable residues in the C-terminal tail were not found to regulate kinase activity or ATP binding in the present disclosure. In yet another embodiment of this aspect, which may be combined with any of the preceding embodiments, the modified plant SYMRK polypeptide constitutively induces symbiotic organogenesis or induces symbiotic organogenesis in the absence of rhizobial bacteria and/or arbuscular mycorrhizal fungi recognized by the plant at a higher level than the unmodified plant SYMRK polypeptide under the same conditions. In an additional embodiment of this aspect, the substitution of step (i) is sufficient to drive symbiotic signaling without rhizobial bacteria and/or arbuscular mycorrhizal fungi recognized by the plant and/or sufficient to drive nodule organogenesis independently of Nod factor receptors (e.g., NFR1/NFR5). In yet another embodiment of this aspect, the Nod factor receptors are needed to synchronize organogenesis and infection processes in nitrogen-fixing nodulation symbiosis. In a further embodiment of this aspect, the organogenesis is nodule formation, arbuscule or vesicle formation, or lateral root formation, preferably nodule formation. In yet another embodiment of this aspect, which may be combined with any preceding embodiment that has the modified plant SYMRK polypeptide constitutively inducing symbiotic organogenesis, the symbiosis is nitrogen-fixing nodulation symbiosis or arbuscular mycorrhizal symbiosis, preferably nitrogen-fixing nodulation symbiosis. In a further embodiment, the modified plant SYMRK polypeptide comprises an active kinase domain.

### Genetically Modified Plants and Related Methods

Some aspects of the disclosure include a genetically modified plant or part thereof including the modified plant SYMRK receptor polypeptide of any one of the preceding embodiments. In a further embodiment of this aspect, the modified plant SYMRK polypeptide constitutively induces symbiotic organogenesis or induces symbiotic organogenesis in the absence of rhizobial bacteria and/or arbuscular mycorrhizal fungi recognized by the plant at a higher level than the unmodified plant SYMRK polypeptide under the same conditions. In still another embodiment of this aspect, which may be combined with any of the preceding embodiments, the organogenesis is nodule formation, arbuscule or vesicle formation, or lateral root formation, preferably nodule formation. In yet another embodiment of this aspect, which may be combined with any of the preceding embodiments, the symbiosis is nitrogen-fixing nodulation symbiosis or arbuscular mycorrhizal symbiosis, preferably nitrogen-fixing nodulation symbioses. In a further embodiment, the modified plant SYMRK polypeptide comprises an active kinase domain. An additional embodiment of this aspect, which may be combined with any of the preceding embodiments, further includes a genetically modified plant NFR1 LysM receptor polypeptide and/or a genetically modified plant NFR5 LysM receptor polypeptide, which can include, without limitation, a transfected plant NFR1 LysM receptor polypeptide encoding nucleic acid, a transfected plant NFR5 LysM receptor polypeptide encoding nucleic acid, an endogenous LysM receptor polypeptide modified for NFR1 nod-factor recognition and/or signaling, and/or an endogenous LysM receptor polypeptide modified for NFR5 nod-factor recognition and/or signaling. The NFR1 and/or NFR5 polypeptide may be from a legume or a non-legume plant species. In a further embodiment of this aspect, the genetically modified plant NFR1 LysM receptor polypeptide and/or genetically modified plant NFR5 LysM receptor polypeptide are modified as described in U.S. patent application Ser. No. 17/265,793, U.S. patent application Ser. No. 17/267,240, U.S. patent application Ser. No. 17/324,354, and/or Rübsam et al., Science (2023) 379 (6629): 272-277.

Additional aspects of the disclosure relate to methods of producing the genetically modified plant or part thereof of any of the preceding embodiments, including introducing a genetic alteration to the plant including a first nucleic acid sequence encoding the modified plant SYMRK receptor polypeptide. In a further embodiment of this aspect, the nucleic acid sequence is operably linked to a promoter, wherein the promoter is a root active promoter, an inducible promoter, a constitutive promoter, or a combination thereof. In still another embodiment of this aspect, the promoter is a root active promoter, and wherein the promoter is selected from the group of a NFR1 promoter, a NFR5 promoter, a LYK3 promoter, a CERK6 promoter, a NFP promoter, a Lotus japonicus NFR5 promoter (SEQ ID NO: 9; UniProt ID Q70KR1), a Lotus japonicus NFR1 promoter (SEQ ID NO: 10; UniProt ID E6YDV0), a Lotus japonicus CERK6 promoter (SEQ ID NO: 11; UniProt ID D3KTZ6), a Medicago truncatula NFP promoter (SEQ ID NO: 12; UniProt ID QOGXS4), a Medicago truncatula LYK3 promoter (SEQ ID NO: 13; UniProt ID Q6UD73), a maize allothioneine promoter, a chitinase promoter, a maize ZRP2 promoter, a tomato LeExtl promoter, a glutamine synthetase soybean root promoter, a RCC3 promoter, a rice antiquitine promoter, a LRR receptor kinase promoter, or an Arabidopsis pCO2 promoter. In a further embodiment of this aspect, the promoter is a constitutive promoter, and wherein the promoter is selected from the group of a CaMV35S promoter, a derivative of the CaMV35S promoter, a maize ubiquitin promoter, a trefoil promoter, a vein mosaic cassava virus promoter, or an Arabidopsis UBQ10 promoter. In yet another embodiment of this aspect, the nucleic acid sequence is inserted into the genome of the plant so that the nucleic acid sequence is operably linked to an endogenous promoter, and wherein the endogenous promoter is a root active promoter. An additional embodiment of this aspect, which may be combined with any of the preceding embodiments, further includes introducing one or more genetic alterations to the plant comprising a second nucleic acid sequence encoding a modified plant NFR1 LysM receptor polypeptide and/or a third nucleic acid sequence encoding a modified plant NFR5 LysM receptor polypeptide.

Further aspects of the disclosure relate to methods of producing the genetically modified plant or part thereof of any of the preceding embodiments, including genetically modifying the plant or part thereof by transforming the plant or part thereof with one or more gene editing components that target an endogenous nuclear genome sequence encoding an endogenous plant SYMRK receptor polypeptide and introduce (i) the substitution of one or more, two or more, three or more, or all four phosphorylatable amino acid residues corresponding to amino acids S877, S885, S889 and S893 of SEQ ID NO: 2 (LjSYMRK UniProt ID Q8LKX1) with a phosphomimetic amino acid residue, and/or (ii) the substitution of one or more, two or more, three or more, four or more, or all five phosphorylatable amino acid residues corresponding to amino acids S724, S731, S742, S751, or S754 of SEQ ID NO: 2 (LjSYMRK UniProt ID Q8LKX1) with a non-phosphorylatable amino acid residue. In an additional embodiment of this aspect, the one or more gene editing components include a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence. Yet another embodiment of this aspect, which may be combined with any of the preceding embodiments, further includes genetically modifying the plant or part thereof by transforming the plant or part thereof with one or more gene editing components that target a second endogenous nuclear genome sequence encoding an endogenous plant NFR1 LysM receptor polypeptide for modification and/or that target a third endogenous nuclear genome sequence encoding an endogenous plant NFR5 LysM receptor polypeptide for modification.

In a further embodiment of the preceding aspects, which may be combined with any of the preceding embodiments that has a genetically modified plant or part thereof, the plant part is a leaf, a stem, a root, a root primordia, a flower, a seed, a fruit, a kernel, a grain, a cell, or a portion thereof. In yet another embodiment of the preceding aspects, which may be combined with any of the preceding embodiments, the plant is selected from the group of cassava (e.g., manioc, yucca, Manihot esculenta), yam (e.g., Dioscorea rotundata, Dioscorea alata, Dioscorea trifida, Dioscorea sp.), sweet potato (e.g., Ipomoea batatas), taro (e.g., Colocasia esculenta), oca (e.g., Oxalis tuberosa), corn (e.g., maize, Zea mays), rice (e.g., indica rice, japonica rice, aromatic rice, glutinous rice, Oryza sativa, Oryza glaberrima), wild rice (e.g., Zizania spp., Porteresia spp.), barley (e.g., Hordeum vulgare), sorghum (e.g., Sorghum bicolor), millet (e.g., finger millet, fonio millet, foxtail millet, pearl millet, barnyard millets, Eleusine coracana, Panicum sumatrense, Panicum milaceum, Setaria italica, Pennisetum glaucum, Digitaria spp., Echinocloa spp.), teff (e.g., Eragrostis tef), oat (e.g., Avena sativa), triticale (e.g., X Triticosecale Wittmack, Triticosecale schlanstedtense Wittm., Triticosecale neoblaringhemii A. Camus, Triticosecale neoblaringhemii A. Camus), rye (e.g., Secale cereale, Secale cereanum), wheat (e.g., common wheat, spelt, durum, cinkorn, emmer, kamut, Triticum aestivum, Triticum spelta, Triticum durum, Triticum urartu, Triticum monococcum, Triticum turanicum, Triticum spp.), Trema spp. (e.g., Trema cannabina, Trema cubense, Trema discolor, Trema domingensis, Trema integerrima, Trema lamarckiana, Trema micrantha, Trema orientalis, Trema philippinensis, Trema strigilosa, Trema tomentosa, Trema levigata), apple (e.g., Malus domestica, Malus pumila, Pyrus malus), pear (e.g., Pyrus communis, Pyrusxbretschneideri, Pyrus pyrifolia, Pyrus sinkiangensis, Pyrus pashia, Pyrus spp.), plum (e.g., Mirabelle, greengage, damson, Prunus domestica, Prunus salicina, Prunus mume), apricot (e.g., Prunus armeniaca, Prunus brigantine, Prunus mandshurica), peach (e.g., Prunus persica), almond (e.g., Prunus dulcis, Prunus amygdalus), walnut (e.g., Persian walnut, English walnut, black walnut, Juglans regia, Juglans nigra, Juglans cinerea, Juglans californica), strawberry (e.g., Fragaria x ananassa, Fragaria chiloensis, Fragaria virginiana, Fragaria vesca), raspberry (e.g., European red raspberry, black raspberry, Rubus idaeus L., Rubus occidentalis, Rubus strigosus), blackberry (e.g., evergreen blackberry, Himalayan blackberry, Rubus fruticosus, Rubus ursinus, Rubus laciniatus, Rubus argutus, Rubus armeniacus, Rubus plicatus, Rubus ulmifolius, Rubus allegheniensis, Rubus subgenus Eubatus sect. Moriferi & Ursini), red currant (e.g., white currant, Ribes rubrum), black currant (e.g., cassis, Ribes nigrum), gooseberry (e.g., Ribes uva-crispa, Ribes grossulari, Ribes hirtellum), melon (e.g., watermelon, winter melon, casabas, cantaloupe, honeydew, muskmelon, Citrullus lanatus, Benincasa hispida, Cucumis melo, Cucumis melo cantalupensis, Cucumis melo inodorus, Cucumis melo reticulatus), cucumber (e.g., slicing cucumbers, pickling cucumbers, English cucumber, Cucumis sativus), pumpkin (e.g., Cucurbita pepo, Cucurbita maxima), squash (e.g., gourd, Cucurbita argyrosperma, Cucurbita ficifolia, Cucurbita maxima, Cucurbita moschata), grape (e.g., Vitis vinifera, Vitis amurensis, Vitis labrusca, Vitis mustangensis, Vitis riparia, Vitis rotundifolia), bean (e.g., Phaseolus vulgaris, Phaseolus lunatus, Vigna angularis, Vigna radiata, Vigna mungo, Phaseolus coccineus, Vigna umbellate, Vigna acontifolia, Phaseolus acutifolius, Vicia faba, Vicia faba equine, Phaseolus spp., Vigna spp.), soybean (e.g., soy, soya bean, Glycine max, Glycine soja), pca (e.g., Pisum spp., Pisum sativum var. sativum, Pisum sativum var. arvense), pea (e.g., Pisum spp., Pisum sativum var. sativum, Pisum sativum var. arvense), chickpea (e.g., garbanzo, Bengal gram, Cicer arietinum), cowpca (e.g., Vigna unguiculata), pigeon pca (e.g., Arhar/Toor, cajan pea, Congo bean, gandules, Caganus cajan), lentil (e.g., Lens culinaris), Bambara groundnut (e.g., earth pea, Vigna subterranea), lupin (e.g., Lupinus spp.), pulses (e.g., minor pulses, Lablab purpureaus, Canavalia ensiformis, Canavalia gladiate, Psophocarpus tetragonolobus, Mucuna pruriens var. utilis, Pachyrhizus erosus), Medicago spp. (e.g., Medicago sativa, Medicago truncatula, Medicago arborea), Lotus spp. (e.g., Lotus japonicus), forage legumes (e.g., Leucaena spp., Albizia spp., Cyamopsis spp., Sesbania spp., Stylosanthes spp., Trifolium spp., Vicia spp.), indigo (e.g., Indigofera spp., Indigofera tinctoria, Indigofera suffruticosa, Indigofera articulata, Indigofera oblongifolia, Indigofera aspalthoides, Indigofera suffruticosa, Indigofera arrecta), legume trees (e.g., locust trees, Gleditsia spp., Robinia spp., Kentucky coffeetree, Gymnocladus dioicus, Acacia spp., Laburnum spp., Wisteria spp.), or hemp (e.g., cannabis, Cannabis sativa).

In certain embodiments, the plant part may be a seed, pod, fruit, leaf, flower, stem, root, any part of the foregoing or a cell thereof, or a non-regenerable part or cell of a genetically modified plant part. As used in this context, a “non-regenerable” part or cell of a genetically modified plant or part thereof is a part or cell that itself cannot be induced to form a whole plant or cannot be induced to form a whole plant capable of sexual and/or asexual reproduction. In certain embodiments, the non-regenerable part or cell of the plant part is a part of a transgenic seed, pod, fruit, leaf, flower, stem or root or is a cell thereof.

Processed plant products that contain a detectable amount of a nucleotide segment, expressed RNA, and/or protein comprising a genetic modification disclosed herein are also provided. Such processed products include, but are not limited to, plant biomass, oil, meal, animal feed, flour, flakes, bran, lint, hulls, and processed seed. The processed product may be non-regenerable. The plant product can comprise commodity or other products of commerce derived from a transgenic plant or transgenic plant part, where the commodity or other products can be tracked through commerce by detecting a nucleotide segment, expressed RNA, and/or protein that comprises distinguishing portions of a genetic modification disclosed herein.

A control as described herein can be a control sample or a reference sample from a wild-type, an azygous, or a null-segregant plant, species, or sample or from populations thereof. A reference value can be used in place of a control or reference sample, which was previously obtained from a wild-type, azygous, or null-segregant plant, species, or sample or from populations thereof or a group of a wild-type, azygous, or null-segregant plant, species, or sample. A control sample or a reference sample can also be a sample with a known amount of a detectable composition or a spiked sample.

### Expression Vectors, Isolated DNA Molecules, or Recombinant Nucleic Acids; Cells, Compositions, or Kits Including the Same; and Related Methods

Still further aspects of the disclosure relate to an expression vector, isolated DNA molecule, or recombinant nucleic acid including a nucleic acid sequence the modified plant SYMRK receptor polypeptide of any one of the preceding embodiments, optionally operably linked to at least one expression control sequence. In further embodiments of this aspect, the at least one expression control sequence includes a promoter selected from the group of a root active promoter, a constitutive promoter, and a combination thereof. In additional embodiments of this aspect, the promoter is a root active promoter, and wherein the promoter is selected from the group of a NFR1 promoter, a NFR5 promoter, a LYK3 promoter, a CERK6 promoter, a NFP promoter, a Lotus japonicus NFR5 promoter (SEQ ID NO: 9; UniProt ID Q70KR1), a Lotus japonicus NFR1 promoter (SEQ ID NO: 10; UniProt ID E6YDV0), a Lotus japonicus CERK6 promoter (SEQ ID NO: 11; UniProt ID D3KTZ6), a Medicago truncatula NFP promoter (SEQ ID NO: 12; UniProt ID QOGXS4), a Medicago truncatula LYK3 promoter (SEQ ID NO: 13; UniProt ID Q6UD73), a maize allothioneine promoter, a chitinase promoter, a maize ZRP2 promoter, a tomato LeExtl promoter, a glutamine synthetase soybean root promoter, a RCC3 promoter, a rice antiquitine promoter, a LRR receptor kinase promoter, or an Arabidopsis pCO2 promoter. In still further embodiments of this aspect, the promoter is constitutive promoter, and wherein the promoter is selected from the group of a CaMV35S promoter, a derivative of the CaMV35S promoter, a maize ubiquitin promoter, a trefoil promoter, a vein mosaic cassava virus promoter, or an Arabidopsis UBQ10 promoter.

Yet further aspects of the disclosure relate to a bacterial cell or an Agrobacterium cell including the expression vector, isolated DNA molecule, or recombinant nucleic acid of any one of the preceding embodiments.

Additional aspects of the disclosure relate to a genetically modified plant, plant part, plant cell, or seed including the expression vector, isolated DNA molecule, or recombinant nucleic acid of any one of the preceding embodiments.

Further aspects of the disclosure relate to a composition or kit including the expression vector, isolated DNA molecule, or recombinant nucleic acid of any one of the preceding embodiments, the bacterial cell or Agrobacterium cell of the preceding embodiments, or the genetically modified plant, plant part, plant cell, or seed of the preceding embodiments.

Yet further aspects of the disclosure relate to a non-regenerable part or cell of the genetically modified plant or part thereof of any one of the preceding embodiments.

Still another aspect of the disclosure relates to methods of constitutively inducing symbiotic organogenesis or inducing symbiotic organogenesis in the absence of rhizobial bacteria and/or arbuscular mycorrhizal fungi recognized by the plant including introducing a genetic alteration via the expression vector, isolated DNA molecule, or recombinant nucleic acid of any one of the preceding embodiments.

### Symbiosis Receptor Kinase (SYMRK)

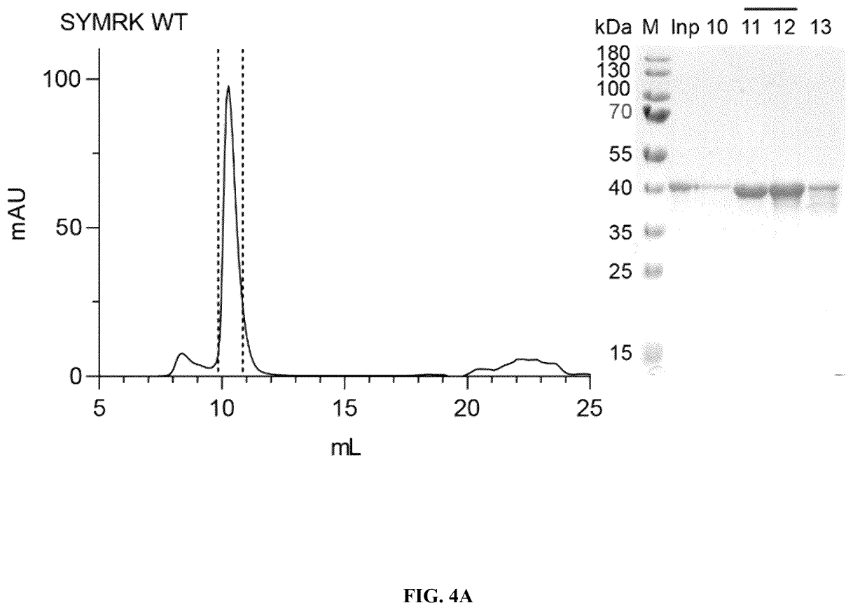

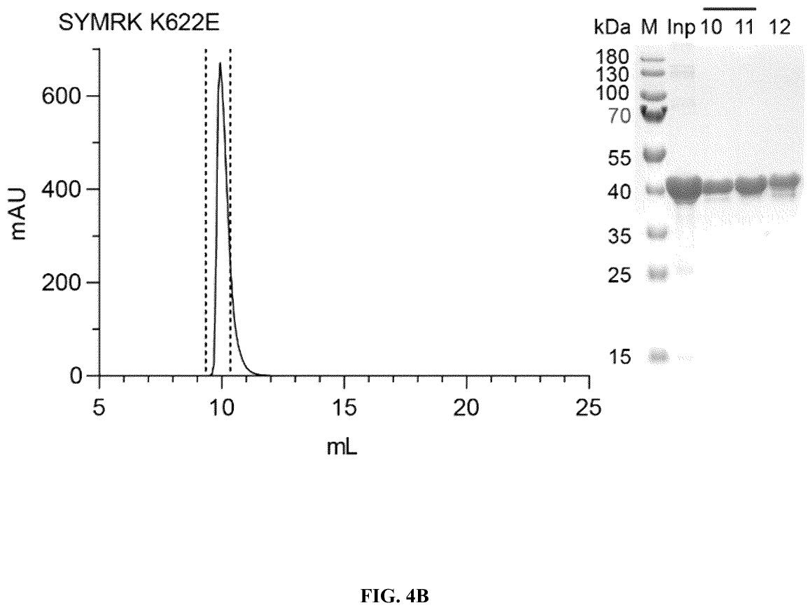

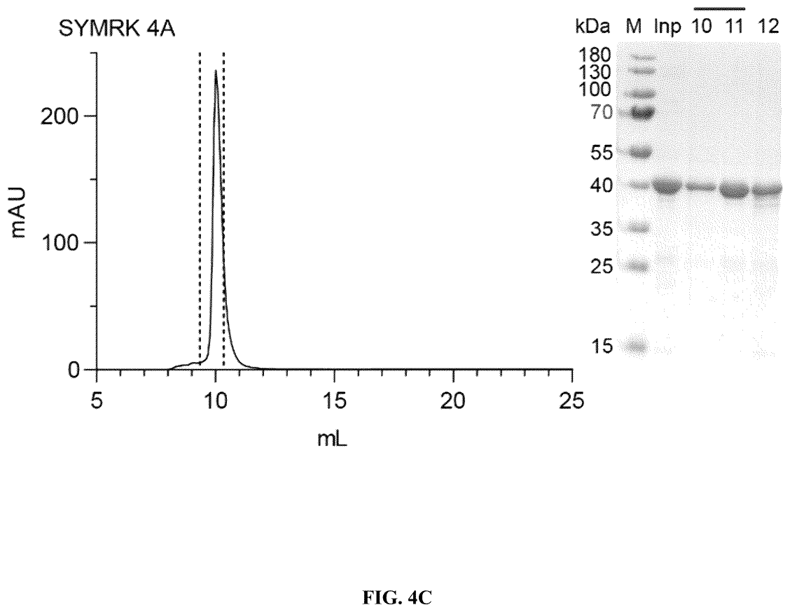

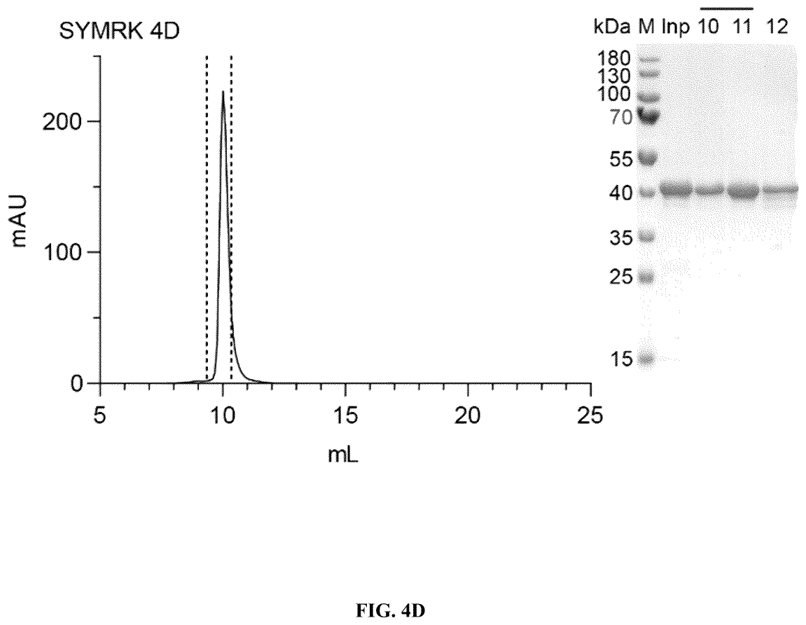

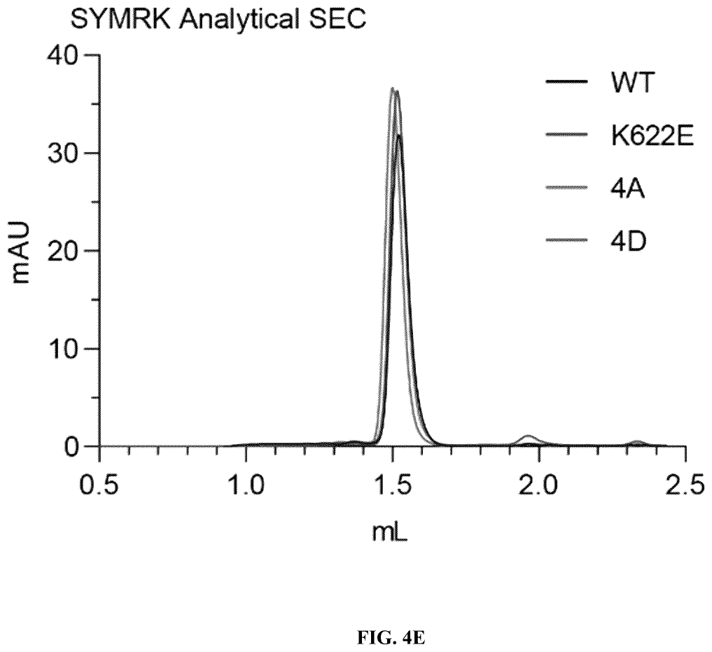

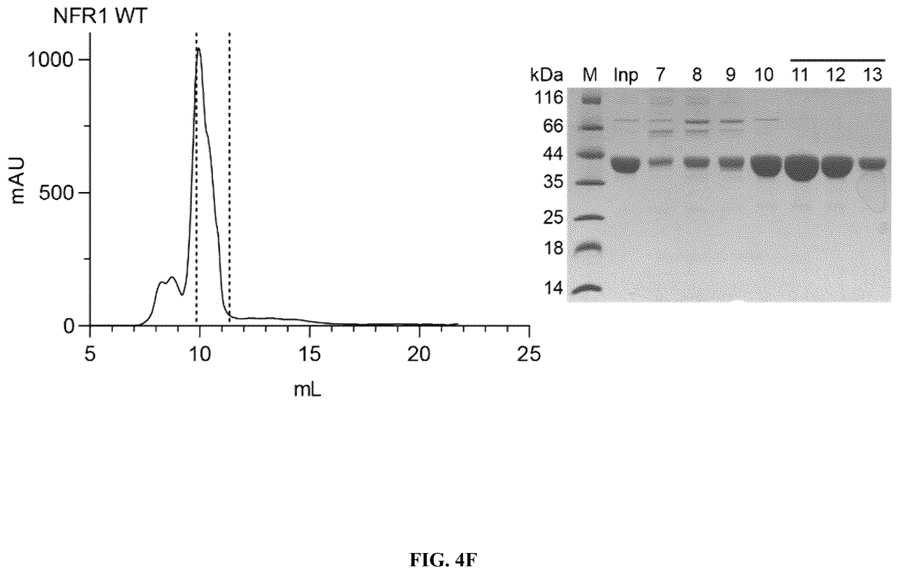

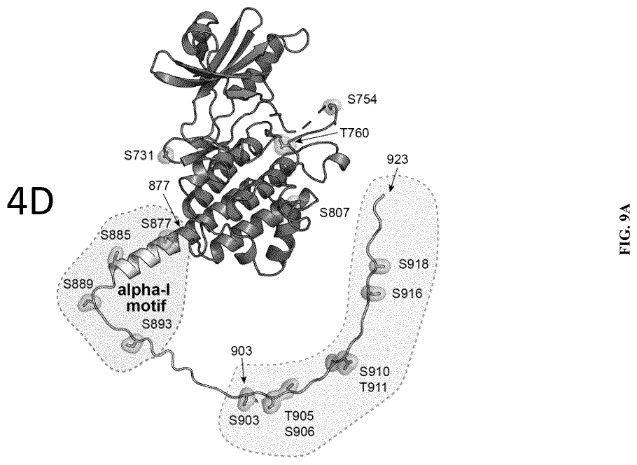

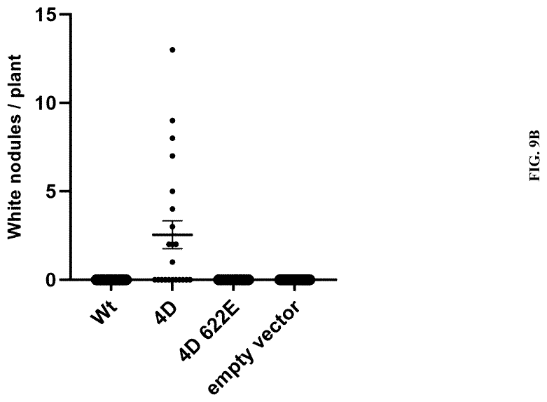

SYMRK is a Malectin-like/leucine-rich repeat receptor kinase identified in 2002 (Stracke et al., Nature. (2002) 417 (6892): 959-62). SYMRK is an active kinase located at the plant cell membrane, and is the first step in the common symbiotic pathway. Specifically, it is involved in both rhizobial nodule symbiosis (RNS) and arbuscular mycorrhizal symbiosis (AMS) and is therefore conserved among most land plants (Markmann et al., PLOS Biol (2008), 6:0497-0506). The present disclosure identifies 20 phosphorylation sites found in two regions of SYMRK, namely a region around the core kinase and a region at the C-terminal tail. At the C-terminal tail of SYMRK, the residues closest to the kinase domain are the most conserved; this includes the S877, S885, S889, and S893 residues (FIGS. 4A-4F) that were identified to be both necessary and sufficient to drive organogenesis. The kinase activity of SYMRK is necessary for spontaneous nodulation. The present disclosure found that a SYMRK mutant without kinase activity did not complement the loss of nodulation phenotype in a null symrk-3 mutant (FIG. 9B).

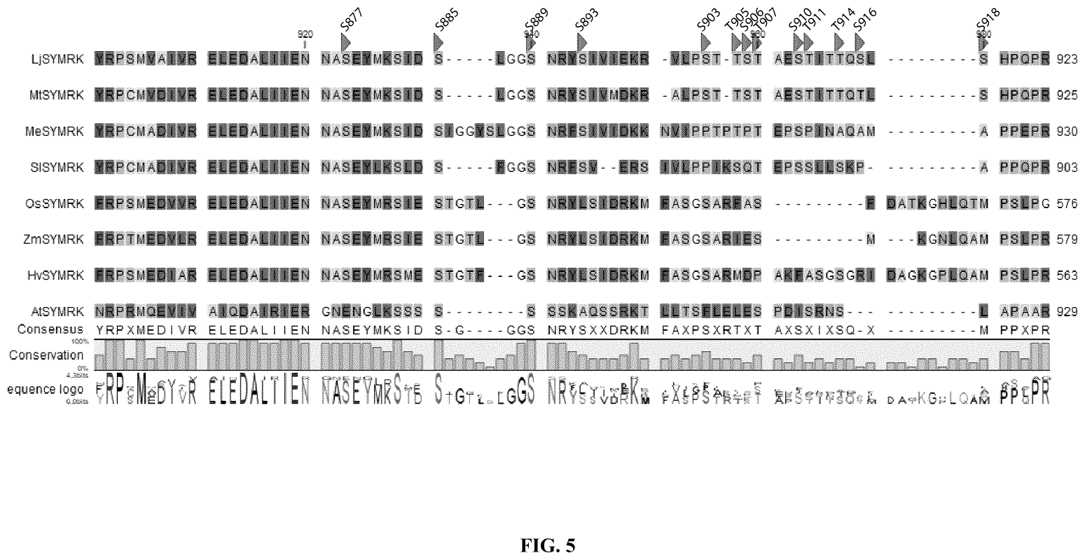

These conserved four residues (S877, S885, S889, and S893) are conserved in legumes and monocot crops (e.g., rice, maize, cassava), but not in Arabidopsis (FIG. 5). It has been shown that symrk mutants of Lotus can be complemented by SYMRK from Medicago as well as by non-nodulating species like rice (Markmann et al., PLOS Biol (2008), 6:0497-0506, Li et al., Front Plant Sci (2018) 9). On the other hand, SYMRK from Arabidopsis cannot complement symrk mutants of Lotus (Li et al., Front Plant Sci (2018) 9). Arabidopsis SYMRK, contrary to Lotus, Medicago and rice SYMRK, lacks two out of the four serines identified in this study to be important for nodulation.

Exemplary SYMRK sequences of the present disclosure include the following: Lotus japonicus (Lj; full length sequence=SEQ ID NO: 2; UniProt ID Q8LKX1), Medicago truncatula (Mt; full length sequence=SEQ ID NO: 4; UniProt ID Q8L4H4), Zea mays (Zm; full length sequence=SEQ ID NO: 1; UniProt ID Q208N5), Oryza sativa (Os; full length sequence=SEQ ID NO: 6; UniProt ID Q7F110), Solanum lycopersicum (S1; full length sequence=SEQ ID NO: 3; UniProt ID Q2TDW9), Manihot esculenta (Me; full length sequence=SEQ ID NO: 5; UniProt ID A0A2C9UVV4), Hordeum vulgare (Hv; full length sequence=SEQ ID NO: 7; UniProt ID A0A816XF58) and Arabidopsis thaliana (At; full length sequence=SEQ ID NO: 8; UniProt ID COLGI2).

### Plant Breeding Methods

Plant breeding begins with the analysis of the current germplasm, the definition of problems and weaknesses of the current germplasm, the establishment of program goals, and the definition of specific breeding objectives. The next step is the selection of germplasm that possess the traits to meet the program goals. The selected germplasm is crossed in order to recombine the desired traits and through selection, varieties or parent lines are developed. The goal is to combine in a single variety or hybrid an improved combination of desirable traits from the parental germplasm. These important traits may include higher yield, field performance, improved fruit and agronomic quality, resistance to biological stresses, such as diseases and pests, and tolerance to environmental stresses, such as drought and heat.

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.). Promising advanced breeding lines are thoroughly tested and compared to appropriate standards in environments representative of the commercial target area(s) for three years at least. The best lines are candidates for new commercial cultivars; those still deficient in a few traits are used as parents to produce new populations for further selection. These processes, which lead to the final step of marketing and distribution, usually take five to ten years from the time the first cross or selection is made.

The choice of breeding or selection methods depends on the mode of plant reproduction, the heritability of the trait(s) being improved, and the type of cultivar used commercially (e.g., F₁ hybrid cultivar, inbred cultivar, etc.). For highly heritable traits, a choice of superior individual plants evaluated at a single location will be effective, whereas for traits with low heritability, selection should be based on mean values obtained from replicated evaluations of families of related plants. The complexity of inheritance also influences the choice of the breeding method. Backcross breeding is used to transfer one or a few genes for a highly heritable trait into a desirable cultivar (e.g., for breeding disease-resistant cultivars), while recurrent selection techniques are used for quantitatively inherited traits controlled by numerous genes, various recurrent selection techniques are used. Commonly used selection methods include pedigree selection, modified pedigree selection, mass selection, and recurrent selection.

Pedigree selection is generally used for the improvement of self-pollinating crops or inbred lines of cross-pollinating crops. Two parents which possess favorable, complementary traits are crossed to produce an F₁. An F₂ population is produced by selfing one or several F₁s or by intercrossing two F₁s (sib mating). Selection of the best individuals is usually begun in the F₂ population; then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Mass and recurrent selections can be used to improve populations of either self-or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding (i.e., recurrent selection) may be used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs, which are also referred to as Microsatellites), Fluorescently Tagged Inter-simple Sequence Repeats (ISSRs), Single Nucleotide Polymorphisms (SNPs), Genotyping by Sequencing (GbS), and Next-generation Sequencing (NGS).

Molecular markers, or “markers”, can also be used during the breeding process for the selection of qualitative traits. For example, markers closely linked to alleles or markers containing sequences within the actual alleles of interest can be used to select plants that contain the alleles of interest. The use of markers in the selection process is often called genetic marker enhanced selection or marker-assisted selection. Methods of performing marker analysis are generally known to those of skill in the art.

Mutation breeding may also be used to introduce new traits into plant varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development: Theory and Technique, Walter Fehr (1991), Agronomy Books, 1 (https://lib.dr.iastate.edu/agron_books/1).

The production of double haploids can also be used for the development of homozygous lines in a breeding program. Double haploids are produced by the doubling of a set of chromosomes from a heterozygous plant to produce a completely homozygous individual. For example, see Wan, et al., Theor. Appl. Genet., (1989) 77:889-892.

Additional non-limiting examples of breeding methods that may be used include, without limitation, those found in Principles of Plant Breeding, John Wiley and Son, pp. 115-161 (1960); Principles of Cultivar Development: Theory and Technique, Walter Fehr (1991), Agronomy Books, 1 (https://lib.dr.iastate.edu/agron_books/1), which are herewith incorporated by reference.

### Molecular Biological Methods to Produce Transgenic Plant Cells, Plant Parts, and Plants

One aspect of the present disclosure provides transgenic plant cells, plant parts, or plants including the modified plant SYMRK receptor polypeptide of any one of the preceding embodiments.

Transformation and generation of genetically altered monocotyledonous and dicotyledonous plant cells is well known in the art. Sec, e.g., Weising, et al., Ann. Rev. Genet. 22:421-477 (1988); U.S. Pat. No. 5,679,558; Agrobacterium Protocols, ed: Gartland, Humana Press Inc. (1995); Wang, et al. Acta Hort. 461:401-408 (1998), and Broothaerts, et al. Nature 433:629-633 (2005). The choice of method varies with the type of plant to be transformed, the particular application, and/or the desired result. The appropriate transformation technique is readily chosen by the skilled practitioner.

Any methodology known in the art to delete, insert or otherwise modify the cellular DNA (e.g., genomic DNA and organelle DNA) can be used in practicing the compositions, methods, and processes disclosed herein. As an example, the CRISPR/Cas-9 system and related systems (e.g., TALEN, ZFN, ODN, etc.) may be used to insert a heterologous gene to a targeted site in the genomic DNA or substantially edit an endogenous gene to express the heterologous gene or to modify the promoter to increase or otherwise alter expression of an endogenous gene through, for example, removal of repressor binding sites or introduction of enhancer binding sites. For example, a disarmed Ti plasmid, containing a genetic construct for deletion or insertion of a target gene, in Agrobacterium tumefaciens can be used to transform a plant cell, and thereafter, a transformed plant can be regenerated from the transformed plant cell using procedures described in the art, for example, in EP 0116718, EP 0270822, PCT publication WO 84/02913 and published European Patent application (“EP”) 0242246. Ti-plasmid vectors each contain the gene between the border sequences, or at least located to the left of the right border sequence, of the T-DNA of the Ti-plasmid. Of course, other types of vectors can be used to transform the plant cell, using procedures such as direct gene transfer (as described, for example in EP 0233247), pollen mediated transformation (as described, for example in EP 0270356, PCT publication WO 85/01856, and U.S. Pat. No. 4,684,611), plant RNA virus-mediated transformation (as described, for example in EP 0 067 553 and U.S. Pat. No. 4,407,956), liposome-mediated transformation (as described, for example in U.S. Pat. No. 4,536,475), and other methods such as the methods for transforming certain lines of corn (e.g., U.S. Pat. No. 6,140,553; Fromm et al., Bio/Technology (1990) 8, 833-839); Gordon-Kamm et al., The Plant Cell, (1990) 2, 603-618), rice (Shimamoto et al., Nature, (1989) 338, 274-276; Datta et al., Bio/Technology, (1990) 8, 736-740), and the method for transforming monocots generally (PCT publication WO 92/09696). For cotton transformation, the method described in PCT patent publication WO 00/71733 can be used. For soybean transformation, reference is made to methods known in the art, e.g., Hinchec et al. (Bio/Technology, (1988) 6, 915) and Christou et al. (Trends Biotech, (1990) 8, 145) or the method of WO 00/42207.

Genetically altered plants of the present disclosure can be used in a conventional plant breeding scheme to produce more genetically altered plants with the same characteristics, or to introduce the genetic alteration(s) in other varieties of the same or related plant species. Seeds, which are obtained from the altered plants, preferably contain the genetic alteration(s) as a stable insert in chromosomal DNA or as modifications to an endogenous gene or promoter. Plants including the genetic alteration(s) in accordance with this disclosure include plants including, or derived from, root stocks of plants including the genetic alteration(s) of this disclosure, e.g., fruit trees or ornamental plants. Hence, any non-transgenic grafted plant parts inserted on a transformed plant or plant part are included in this disclosure.

Genetic alterations of the disclosure, including in an expression vector or expression cassette, which result in the expression of an introduced gene or altered expression of an endogenous gene will typically utilize a plant-expressible promoter. A ‘plant-expressible promoter’ as used herein refers to a promoter that ensures expression of the genetic alteration(s) of this disclosure in a plant cell. Examples of constitutive promoters that are often used in plant cells are the cauliflower mosaic (CaMV) 35S promoter (Kay et al. Science, 236, 4805, 1987), the minimal CaMV 35S promoter (Benfey & Chua, Science, (1990) 250, 959-966), various other derivatives of the CaMV 35S promoter, the figwort mosaic virus (FMV) promoter (Richins, et al., Nucleic Acids Res. (1987) 15:8451-8466) the maize ubiquitin promoter (Christensen & Quail, Transgenic Res, 5, 213-8, 1996), the trefoil promoter (Ljubql, Mackawa et al. Mol Plant Microbe Interact. 21, 375-82, 2008), the vein mosaic cassava virus promoter (International Application WO 97/48819), and the Arabidopsis UBQ10 promoter, Norris et al. Plant Mol. Biol. 21, 895-906, 1993).

Additional examples of promoters directing constitutive expression in plants are known in the art and include: the strong constitutive 35S promoters (the “35S promoters”) of the cauliflower mosaic virus (CaMV), e.g., of isolates CM 1841 (Gardner et al., Nucleic Acids Res, (1981) 9, 2871-2887), CabbB S (Franck et al., Cell (1980) 21, 285-294) and CabbB J I (Hull and Howell, Virology, (1987) 86, 482-493); promoters from the ubiquitin family (e.g., the maize ubiquitin promoter of Christensen et al., Plant Mol Biol, (1992) 18, 675-689), the gos2 promoter (de Pater et al., The Plant J (1992) 2, 834-844), the emu promoter (Last et al., Theor Appl Genet, (1990) 81, 581-588), actin promoters such as the promoter described by An et al. (The Plant J, (1996) 10, 107), the rice actin promoter described by Zhang et al. (The Plant Cell, (1991) 3, 1155-1165); promoters of the figwort mosaic virus (FMV) (Richins, et al., Nucleic Acids Res. (1987) 15:8451-8466), promoters of the Cassava vein mosaic virus (WO 97/48819; Verdaguer et al., Plant Mol Biol, (1998) 37, 1055-1067), the pPLEX series of promoters from Subterranean Clover Stunt Virus (WO 96/06932, particularly the S4 or S7 promoter), an alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the l′ and 2′ genes, respectively, of the T DNA (Velten et al., EMBO J, (1984) 3, 2723-2730).

Alternatively, a plant-expressible promoter can be a tissue-specific promoter, i.e., a promoter directing a higher level of expression in some cells or tissues of the plant, e.g., in root epidermal cells or root cortex cells. In preferred embodiments, LysM receptor promoters will be used. Non-limiting examples include NFR1 promoters, NFR5 promoters, LYK3 promoters, NFP promoters, the Lotus japonicus NFR5 promoter (SEQ ID NO: 9; UniProt ID Q70KR1), the Lotus japonicus NFR1 promoter (SEQ ID NO: 10; UniProt ID E6YDV0), the Lotus japonicus CERK6 promoter (SEQ ID NO: 11; UniProt ID D3KTZ6), the Medicago truncatula NFP promoter (SEQ ID NO: 12; UniProt ID QOGXS4), or the Medicago truncatula LYK3 promoter (SEQ ID NO: 13; UniProt ID Q6UD73). In additional preferred embodiments, root active promoters will be used. Non-limiting examples include the promoter of the maize allothioncine (De Framond et al, FEBS 290, 103-106, 1991 Application EP 452269), the chitinase promoter (Samac et al. Plant Physiol 93, 907-914, 1990), the glutamine synthetase soybean root promoter (Hirel et al. Plant Mol. Biol. 20, 207-218, 1992), the RCC3 promoter (PCT Application WO 2009/016104), the rice antiquitine promoter (PCT Application WO 2007/076115), the LRR receptor kinase promoter (PCT application WO 02/46439), the maize ZRP2 promoter (U.S. Pat. No. 5,633,363), the tomato LeExtl promoter (Bucher et al. Plant Physiol. 128, 911-923, 2002), and the Arabidopsis pCO2 promoter (Heidstra et al, Genes Dev. 18, 1964-1969, 2004). These plant promoters can be combined with enhancer elements, they can be combined with minimal promoter elements, or can comprise repeated elements to ensure the expression profile desired.

Examples of constitutive promoters that are often used in plant cells are the cauliflower mosaic (CaMV) 35S promoter (Kay et al. Science, 236, 4805, 1987), and various derivatives of the promoter, virus promoter vein mosaic cassava (International Application WO 97/48819), the maize ubiquitin promoter (Christensen & Quail, Transgenic Res, 5, 213-8, 1996), trefoil (Ljubql, Mackawa et al. Mol Plant Microbe Interact. 21, 375-82, 2008) and Arabidopsis UBQ10 (Norris et al. Plant Mol. Biol. 21, 895-906, 1993).

In some embodiments, further genetic alterations to increase expression in plant cells can be utilized. For example, an intron at the 5′ end or 3′ end of an introduced gene, or in the coding sequence of the introduced gene, e.g., the hsp70 intron. Other such genetic elements can include, but are not limited to, promoter enhancer elements, duplicated or triplicated promoter regions, 5′ leader sequences different from another transgene or different from an endogenous (plant host) gene leader sequence, 3′ trailer sequences different from another transgene used in the same plant or different from an endogenous (plant host) trailer sequence.

An introduced gene of the present disclosure can be inserted in host cell DNA so that the inserted gene part is upstream (i.e., 5′) of suitable 3′ end transcription regulation signals (i.e., transcript formation and polyadenylation signals). This is preferably accomplished by inserting the gene in the plant cell genome (nuclear or chloroplast). Preferred polyadenylation and transcript formation signals include those of the nopaline synthase gene (Depicker et al., J. Molec Appl Gen, (1982) 1, 561-573), the octopine synthase gene (Gielen et al., EMBO J, (1984) 3:835-845), the SCSV or the Malic enzyme terminators (Schunmann et al., Plant Funct Biol, (2003) 30:453-460), and the T DNA gene 7 (Velten and Schell, Nucleic Acids Res, (1985) 13, 6981-6998), which act as 3′ untranslated DNA sequences in transformed plant cells. In some embodiments, one or more of the introduced genes are stably integrated into the nuclear genome. Stable integration is present when the nucleic acid sequence remains integrated into the nuclear genome and continues to be expressed (i.e., detectable mRNA transcript or protein is produced) throughout subsequent plant generations. Stable integration into the nuclear genome can be accomplished by any known method in the art (e.g., microparticle bombardment, Agrobacterium-mediated transformation, CRISPR/Cas9, electroporation of protoplasts, microinjection, etc.).

The term recombinant or modified nucleic acids refers to polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

As used herein, the term “overexpression” refers to increased expression (e.g., of mRNA, polypeptides, etc.) relative to expression in a wild type organism (e.g., plant) as a result of genetic modification and can refer to expression of heterologous genes at a sufficient level to achieve the desired result such as increased yield. In some embodiments, the increase in expression is a slight increase of about 10% more than expression in wild type. In some embodiments, the increase in expression is an increase of 50% or more (e.g., 60%, 70%, 80%, 100%, etc.) relative to expression in wild type. In some embodiments, an endogenous gene is upregulated. In some embodiments, an exogenous gene is upregulated by virtue of being expressed. Upregulation of a gene in plants can be achieved through any known method in the art, including but not limited to, the use of constitutive promoters with inducible response elements added, inducible promoters, high expression promoters (e.g., PsaD promoter) with inducible response elements added, enhancers, transcriptional and/or translational regulatory sequences, codon optimization, modified transcription factors, and/or mutant or modified genes that control expression of the gene to be upregulated in response to a stimulus such as cytokinin signaling.

Where a recombinant nucleic acid is intended for expression, cloning, or replication of a particular sequence, DNA constructs prepared for introduction into a host cell will typically include a replication system (e.g., vector) recognized by the host, including the intended DNA fragment encoding a desired polypeptide, and can also include transcription and translational initiation regulatory sequences operably linked to the polypeptide-encoding segment. Additionally, such constructs can include cellular localization signals (e.g., plasma membrane localization signals). In preferred embodiments, such DNA constructs are introduced into a host cell's genomic DNA, chloroplast DNA, or mitochondrial DNA.

In some embodiments, a non-integrated expression system can be used to induce expression of one or more introduced genes. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing the methodologies disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present disclosure. One of skill in the art will recognize that any relevant markers available can be utilized in practicing the compositions, methods, and processes disclosed herein.

Screening and molecular analysis of recombinant strains of the present disclosure can be performed utilizing nucleic acid hybridization techniques. Hybridization procedures are useful for identifying polynucleotides, such as those modified using the techniques described herein, with sufficient homology to the subject regulatory sequences to be useful as taught herein. The particular hybridization techniques are not essential to this disclosure. As improvements are made in hybridization techniques, they can be readily applied by one of skill in the art. Hybridization probes can be labeled with any appropriate label known to those of skill in the art. Hybridization conditions and washing conditions, for example, temperature and salt concentration, can be altered to change the stringency of the detection threshold. See, e.g., Sambrook et al. (1989) vide infra or Ausubel et al. (1995) Current Protocols in Molecular Biology, John Wiley & Sons, NY, N.Y., for further guidance on hybridization conditions.

Additionally, screening and molecular analysis of genetically altered strains, as well as creation of desired isolated nucleic acids can be performed using Polymerase Chain Reaction (PCR). PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Because the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

Nucleic acids and proteins of the present disclosure can also encompass homologues of the specifically disclosed sequences. Homology (e.g., sequence identity) can be 50%-100%. In some instances, such homology is greater than 80%, greater than 85%, greater than 90%, or greater than 95% or a functional fragment or conserved domain thereof. The degree of homology or identity needed for any intended use of the sequence(s) is readily identified by one of skill in the art. As used herein percent sequence identity of two nucleic acids is determined using an algorithm known in the art, such as that disclosed by Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN, BLASTP, and BLASTX, programs of Altschul et al. (1990) J. Mol. Biol. 215:402-410. BLAST nucleotide searches are performed with the BLASTN program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (BLASTN and BLASTX) are used. See www.ncbi.nih.gov. One of skill in the art can readily determine in a sequence of interest where a position corresponding to amino acid or nucleic acid in a reference sequence occurs by aligning the sequence of interest with the reference sequence using the suitable BLAST program with the default settings (e.g., for BLASTP: Gap opening penalty: 11, Gap extension penalty: 1, Expectation value: 10, Word size: 3, Max scores: 25, Max alignments: 15, and Matrix: blosum62; and for BLASTN: Gap opening penalty: 5, Gap extension penalty: 2, Nucleic match: 1, Nucleic mismatch-3, Expectation value: 10, Word size: 11, Max scores: 25, and Max alignments: 15).

Preferred host cells are plant cells. Recombinant host cells, in the present context, are those which have been genetically modified to contain an isolated nucleic molecule, contain one or more deleted or otherwise non-functional genes normally present and functional in the host cell, or contain one or more genes to produce at least one recombinant protein. The nucleic acid(s) encoding the protein(s) of the present disclosure can be introduced by any means known to the art which is appropriate for the particular type of cell, including without limitation, transformation, lipofection, electroporation or any other methodology known by those skilled in the art.

“Isolated”, “isolated DNA molecule” or an equivalent term or phrase is intended to mean that the DNA molecule or other moiety is one that is present alone or in combination with other compositions, but altered from or not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” from its natural setting within the scope of this disclosure so long as the element is not within the genome of the organism in which it is naturally found, the element is altered from its natural form, or the element is not at the location within the genome in which it is naturally found. Similarly, a nucleotide sequence encoding a protein or any naturally occurring variant of that protein would be an isolated nucleotide sequence so long as the nucleotide sequence was not within the DNA of the organism from which the sequence encoding the protein is naturally found in its natural location or if that nucleotide sequence was altered from its natural form. A synthetic nucleotide sequence encoding the amino acid sequence of the naturally occurring protein would be considered to be isolated for the purposes of this disclosure. For the purposes of this disclosure, any transgenic nucleotide sequence, i.e., the nucleotide sequence of the DNA inserted into the genome of the cells of a plant, alga, fungus, or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

Having generally described the compositions, methods, and processes of this disclosure, the same will be better understood by reference to certain specific examples, which are included herein to further illustrate the disclosure and are not intended to limit the scope of the invention as defined by the claims.

### EXAMPLES

The present disclosure is described in further detail in the following examples which are not in any way intended to limit the scope of the disclosure as claimed. The attached figures are meant to be considered as integral parts of the specification and description of the disclosure. The following examples are offered to illustrate, but not to limit the claimed disclosure.

### Example 1: Autophosphorylation of SYMRK Drives Root Nodule Organogenesis

The following example describes that symbiosis receptor-like kinase (SYMRK) is required for successful root nodule symbiosis between legume plants and rhizobial bacteria. This study uses mass spectrometry to identify phosphorylatable amino acid residues essential for successful root nodule symbiosis. 20 phosphorylatable amino acid residues found in two regions of SYMRK were identified, one region around the core kinase and one region at the C-terminal tail. It was found that phosphorylation of the C-terminal tail of SYMRK is sufficient to drive organogenesis, as phospho-mimicking of four serine residues resulted in the formation of spontaneous nodules in the absence of symbiotic bacteria. Contrary to this, alanine substitutions of the same four residues completely inhibited the symbiotic pathway without influencing the localization, activity, or the ATP-binding ability of SYMRK.

### Introduction

Root nodule symbiosis is regulated by phosphorylation signaling. It has been shown that correct phosphorylation at the activation loops of Lotus japonicus (Lotus) NFR1 and of its homologue in Medicago truncatula (Medicago), LYK3, has been shown to be required for nodule formation (Madsen et al., Nature Communications, (2010) 1 (10); Madsen et al., Plant J. (2011) 65 (3) 404-417, Klaus-Heisen et al., Journal of Biological Chemistry (2011) 286:11202-11210). Further downstream in the CSP, phosphorylation of CYCLOPS and CCAMK is also essential for nodule development (Singh et al., Cell Host Microbe (2014) 15:139-152, Tirichine et al., Nature (2006) 441:1153-1156). SYMRK in Arachis hypogaea has been shown to be regulated by phosphorylation (Saha et al, Plant Physiol (2016) 171:71-81) and the activity of Lotus SYMRK kinase activity is regulated by phosphorylation at T760 (Yoshida and Parniske, Journal of Biological Chemistry (2004) 280:9203-9209). This example identified novel phosphorylatable amino acid residues in the C-terminal tail of Lotus SYMRK. These phosphorylatable amino acid residues are essential for successful symbiosis but are also sufficient to drive organogenesis in the absence of symbiotic bacteria.

### Materials and Methods

### Plasmid Construction and Cloning

Constructs for expressing phosphomimetic or phosphorylation-null mutants of SYMRK were generated using synthesized modules of the 5 kbp promoter of SymRK, the 300 bp terminator, wild type-, phosphomimetic-or phosphorylation-ablation mutants of SYMRK (Thermo Fisher Scientific). mCherry and L1 vectors used are previously described in Weber et al. PLOS One (2011) 6. Expression vectors used are pIV10 (Radutoiu 2005). All modules are assembled as described in Weber 2011 (Weber et al. PLOS One (2011) 6). The following modules were used: Native promoter of SYMRK from Lotus (pSYMRK) SYMRK gene from Lotus (SYMRK), native terminator of Lotus (tSYMRK), ubiquitin promoter from Lotus (pUBI), Triple YFP (YFP), nuclear localization signal (NLS), Promoter of the cauliflower mosaic virus (p35s) and terminator of the cauliflower mosaic virus (t35s).

Plasmids for Escherichia coli expression of SYMRK kinase domains for crystallization, phosphorylation, and ATP binding were synthesized in pAH10R7Sumo3C (Andersen et al., Elife (2013)) by Genscript. Expression and purification of NFR1 kinase domain was performed as previously described by Wong & Nadzieja 2019 (Wong et al., PNAS (2019) 116 (28) 14339-14348).

Expression and Purification of Protein from E. coli

SYMRK and NFR1 kinase constructs were transformed into E. coli Rosetta 2 and grown in LB until OD600=0.6. Protein expression was induced by addition of 0.4 mM IPTG and cultures were incubated at 18° C. overnight. Protein was captured from cleared E. coli lysate on a Protino Ni-NTA column (Macherey-Nagel) equilibrated in buffer A (25 mM HEPES pH 7.5, 500 mM NaCl, 20 mM imidazole, 5 mM β-mercaptoethanol, 5% glycerol) and eluted in buffer B (buffer A supplemented with 500 mM imidazole). Ni-AC eluates were dialyzed overnight at 4° C. in dialysis buffer (25 mM HEPES pH 7.5, 500 mM NaCl, 1 mM MnCl₂, 5 mM β-mercaptoethanol, 5% glycerol). During dialysis, the samples were de-phosphorylated and cleaved by addition of his-tagged λ-protein phosphatase and protease (3C for SYMRK and TEV for NFR1) in a 1:200 and 1:50 molar ratio, respectively. Dialysates were purified in a second Ni-AC step to remove cleaved fusion-tags and his-tagged enzymes. Protein purification was finalized in two size exclusion chromatography steps using first a Superdex 200 increase 10/300 and second a Superdex 75 increase 10/300 columns (Cytiva) in SEC buffer (25 mM HEPES pH 7.5, 200 mM NaCl, 5 mM β-mercaptocthanol). FIGS. 4A-4F show SEC plots and SDS-PAGE gels for the SYMRK and NFR1 kinase constructs that were transformed into E. coli, which were used as quality control for the proteins produced in E. coli.

### Sample Preparation and Phosphor Enrichment for Mass Spectrometry

Kinases of SYMRK or NFR1 purified from E. coli were buffer exchanged into 50 mM Tris-HCl pH 8, 200 mM NaCl, 2.5 mM DTT by SEC. Phosphorylation reactions were performed in 10 μL reaction mixtures containing 5 μg of each protein and supplemented 5 mM MnCl₂, 10 mM MgCl₂, 20 μM ATP. Reactions were incubated at room temperature for 1 hour before flash freezing in liquid nitrogen. Digest was performed in 250 μL trypsin buffer (50 mM ammonium bicarbonate, 1% SDC pH=8.5) incubated 10 min at 80° C. followed by 10 min on ice and 3% M/M Trypsin/Lys-C Mix (Promega) was added. Digestions were performed at 37° C. shaking for 18 hours in the dark. The digestion was terminated by addition of TFA to 1%. Reaction was spun down for 10 min at 20.000 rpm and supernatant was used for desalting. 200u L tips with C18 membrane and OLIGO R3 Reversed phase resin (Thermo scientific) was employed for desalting, samples were washed twice with 100 μL 1% formic acid and eluted in 50 μL 50% acetonitrile+0.1% formic acid. Eluted peptides were dried using a speedvac and resuspended in 50 μL 0.1% formic acid. Phosphor enrichment was performed using MagReSYN Zr IMAC beads (Resynbio) (10 times binding capacity to protein amount) with a Kingfisher robot (Thermo scientific). Resuspended peptides+MagReSYN Zr IMAC bead were washed with 80% acetonitrile, 5% TFA, 0,1M GA followed by wash with 80% acetonitrile, 1% TFA and finally with 10% acetonitrile, 0.2% TFA and diluted in 1.25M NH₂OH. Samples were dried using a speedvac and resuspended in 12 μL 0.1% formic acid.

### Crystallization and Structure Determination

Crystals of LjSYMRK-D738N (587-877) (i.e., LjSYMRK protein with a D to N mutation in amino acid position 738 and truncation 0-586 and 877-923) were obtained using a sitting drop vapor diffusion system at 5 to 10 mg/ml in 1.26 M ammonium sulphate, 0.1 M Tris-HCl pH 8.5. Crystals were cryoprotected by incremental soaking steps in mother liquor containing 10-30% (v/v) ethylene glycol before snap-freezing in liquid nitrogen. Diffraction data to 1.95 Å resolution were obtained at the DESY P13 beamline in Hamburg, Germany at a wavelength of 1.0 Å. Data reduction and scaling was performed in XDS (Kabsch, Acta Crytallogr D. Biolo Crystallogr (2010) 66:133-144) and XSCALE, respectively. The phase problem was solved by molecular replacement in phenix.phaser (McCoy et al., J Appl Crystallogr (2007) 40:658-674) (Liebschner et al., Acta Crystallogr D Struct Biol (2019) 75:861-877) using a homology model built in SWISS-MODEL (Waterhouse et al., Nucleic Acids Res (2018) 46: W296-W303) based on the crystal structure of AtBIKI kinase domain (PDB: 5TOS) (Lal et al., Cell Host Microbe (2018) 23:485-497.e5) as a search model. The structure was built in Coot (Emsley et al., Acta Crystallogr D Biol Crystallogr (2010) 66:486-501), coordinates and B-factors were refined in phenix.refine (Liebschner et al., Acta Crystallogr D Biol Crystallogr (2019) 75:861-877).

### NanoDSF ATP Binding Assay

E. coli expressed SYMRK proteins in SEC buffer were supplemented with 5 mM MgCl2 or 5 mM MgCl2.1 mM ATP for 15 min on ice before three technical replicates of each sample were loaded into Prometheus NT.48 Series nanoDSF Grade Standard Capillaries (NanoTemper Technologies). ATP binding was assayed by proxy of thermal stabilization in a nano differential scanning fluorimetry (NanoDSF) experiment using a Prometheus Panta (NanoTemper Technologies). SYMRK samples were incubated over a temperature gradient from 25° C. to 95° C., with a 1° C./min increment and protein melting was measured by intrinsic fluorescence emissions at 330/350 nm.

### [γ-32P] Radiolabeled ATP Kinase Assay

Radioactive kinase assays were performed in 10 μL reaction mixtures using 3 μg SYMRK and 3 μg Myelin basic protein (MBP) in SEC buffer. Phosphorylation was started by addition of 5 mM MgCl₂, 100 nCi [γ32-P]-ATP and samples were incubated for 1 h at room temperature. Reactions were stopped by addition of SDS loading dye and 95° C. incubation before samples were assayed by SDS-PAGE. The SDS-PAGE gel was incubated on an Autoradiography Hypercassette (Amersham/Cytiva) overnight and the radiograph was developed using a Typhoon FLA 9500 (Amersham/Cytiva). Analytical SEC was performed using a Superdex 200 3.2/300 (Cytiva) in SEC buffer.

### Plant Materials and Growth Conditions

All hairy root experiments were carried out in symrk-3, nfr1/nfr5/symrk or ccamk-13 mutant backgrounds. Seeds were scarified using sandpaper followed by surface sterilization by 15 min in 1% sodium hypochlorite, afterwards washed 5 times in water and incubated rotating at 4° C. overnight. The following day the seeds were set for germination on wet filter paper for 4 days. Four-day-old seedlings were transferred to square agar plates with Gamborg's B5 nutrient solution (Duchefa Biochemie) and 0.8% Gelrite (Duchefa Biochemie). A. rhizogenes AR1193 carrying the indicated constructs were used for root transformation of six-day-old seedlings by punching the hypocotyl with a syringe needle and placing a drop of bacteria on top of the wound. Seedlings and bacteria were incubated at 21° C. for 16 hours in the dark before growing under 16/8-h light/dark conditions for three weeks. Non-transformed roots were removed, and plants were moved to pots with lightweight expanded clay aggregate (LECA, 2-4 mm; Saint-Gobain Weber A/S) supplemented with B&D nutrient solution (Broughton & Dilworth, 1971). One week after transfer to pots, plants were inoculated with M. loti MAFF303099 expressing DsRED bacteria (Mackawa et al., The Plant Journal (2009) 58:183-194) OD600=0.01 and grown for four weeks before harvest.

Transient Transformation of N. benthamiana

Transformation was performed as described previously (Ochoa-Fernandez et al., Nature Methods (2020) 17 (717-725). In brief: A. tumefaciens bacteria were resuspended in infiltration solution (10 mM MgCl₂, 10 mM MES, 150 μM acetosyringone, pH 5.6) to an OD600=0.1, followed by incubated for two hours. The bacteria solution was infiltrated into the leaves with a blunt end syringe and grown for three days.

### Microscopy

Transgenic roots expressing the indicated SYMRK mutant versions or empty vectors were imaged using a Leica FluoStereo M165FC microscope equipped with a Leica DFC310 FX camera. Expression of SYMRK wild-type, phosphor-ablation and phosphor-mimic mutants fused to mCherry in N. benthamiana were imaged using 561 nm excitation with 571 to 642 nm emission on a Zeiss LSM 780 confocal microscope.

### Sequence Alignment

Sequences of the C-terminal tail of SYMRK from Lotus japonicus, Medicago truncatula, Zea mays, Oryza sativa, Solanum lycopersicum, Manihot esculenta, Hordeum vulgare, and Arabidopsis thaliana were aligned using CLC workbench 22 (QIAGEN).

### Results and Discussion

### Crystal Structure of SYMRK Shows Clustering of Phosphorylatable Amino Acid Residues in the Kinase Core and the C-Terminal Tail

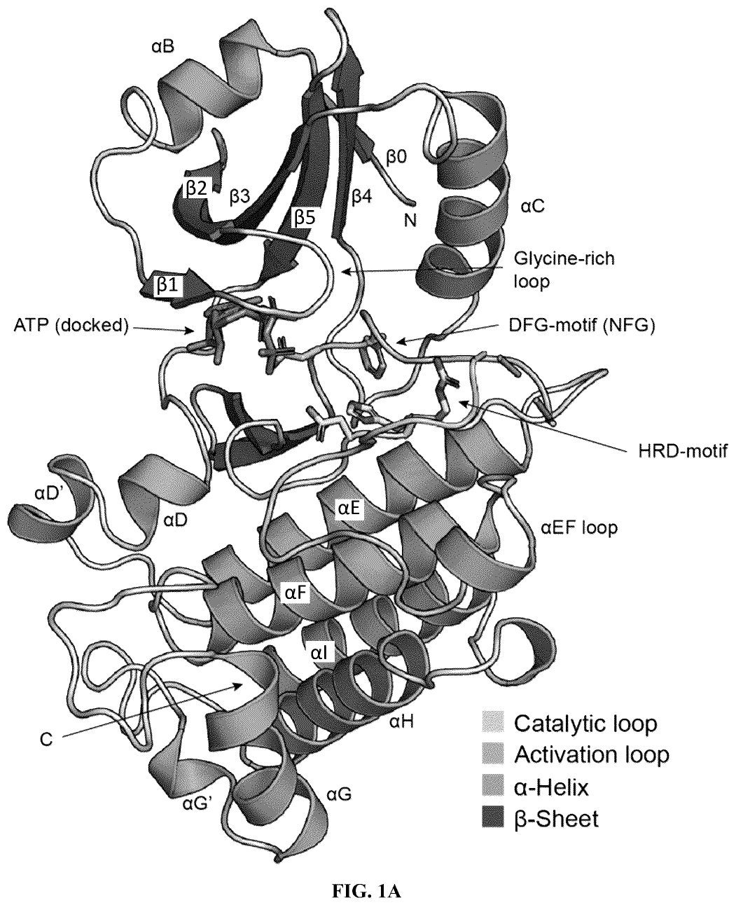

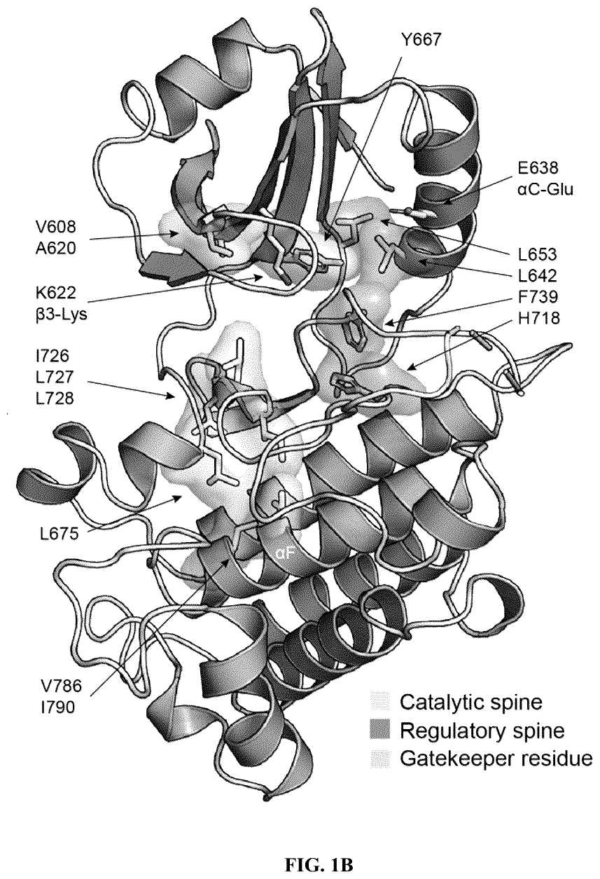

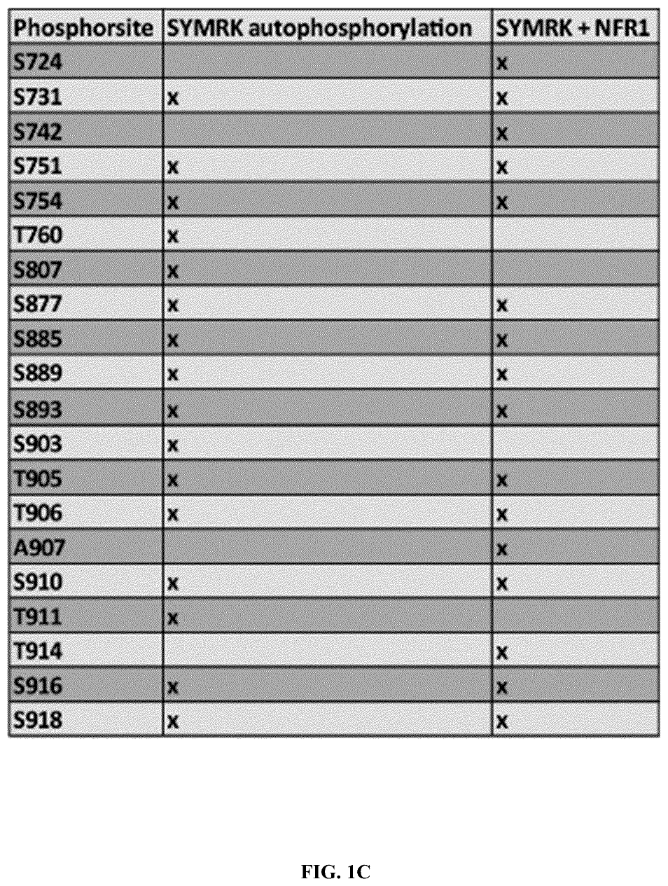

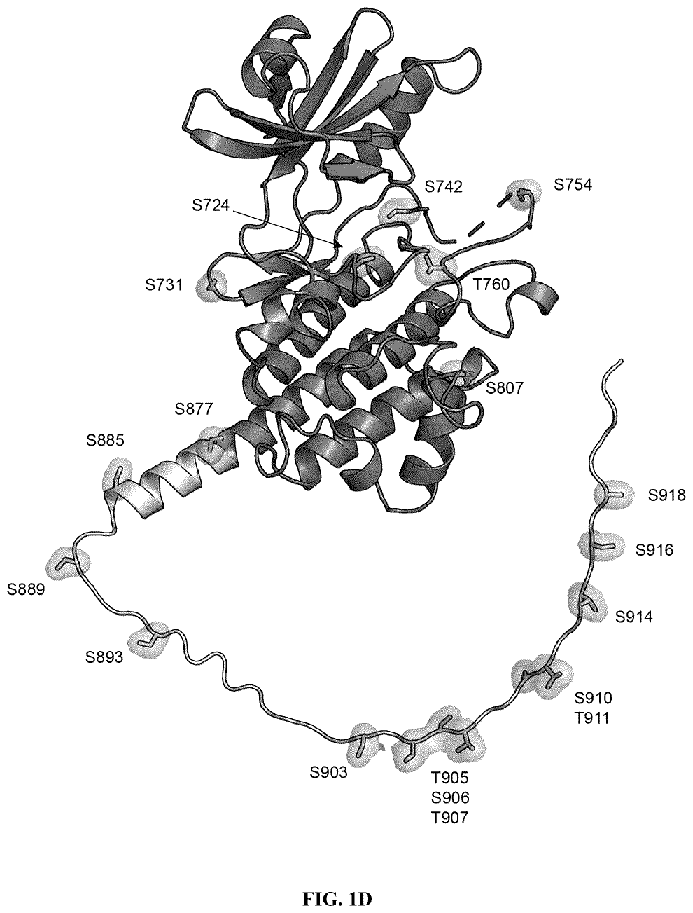

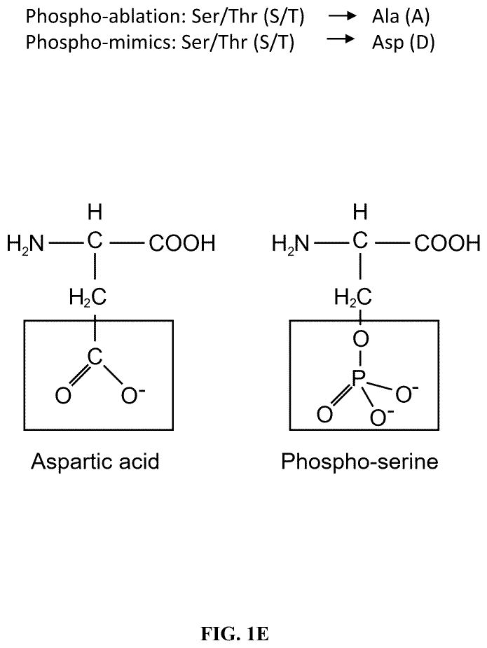

One of the most common ways to regulate complex pathways in plants is by phosphorylation (Jaillais et al., Genes Dev (2011) 25:232-237, Li et al., Cell host Microbe (2014) 15:329-338, Chinchilla et al., Nature (2007) 448:997-500). To gain insights into the regulation of SYMRK, its possible regulation by phosphorylation was investigated. As it has been shown that NFR1 and SYMRK are both active and do interact (Antolin-Llovera, Current Biology (2014) 24:422-427, Radutoiu et al., Nature (2003) 425 (6958): 585-92, Yoshida et al., Journal of Biological Chemistry (2005) 280:9203-9209), the kinase domains of SYMRK and NFR1 were purified and an in vitro phosphorylation assay was conducted. After this, the proteins were digested and the phosphorylated peptides were enriched. Tandem mass spectrometry was used to identify phosphorylated residues on SYMRK. This resulted in the identification of 20 phosphorylatable amino acid residues on SYMRK (FIG. 1C). Sixteen sites were identified as being auto-phosphorylated on SYMRK after incubation with NFR1, and four additional sites (S724, S742, T907 and T914) were further identified (FIG. 1C). Four phosphorylatable amino acid residues were only identified on SYMRK in the absence of NFR1 (T760, S807, S903 and T911). Without wishing to be bound by theory, it is possible that the fact that some phosphorylatable amino acid residues were identified only on SYMRK alone and not in combination with NFR1 could be an effect of multiple phosphorylatable amino acid residues being identified on the same peptides, as well as some sites being present in only very low abundance. This may be due to a general problem of using a mass spectrometry approach to identify phosphorylatable amino acid residues, as mass spectrometry picks out the 20 highest intensity peptides each run, but if the peptide carrying the phosphorylatable amino acid residue is below top 20 it will not be identified in that run.

In order to connect the identified phosphorylatable amino acid residues to biological functions, a fundamental understanding of the SYMRK kinase domain was needed. A soluble SYMRK kinase construct was thus designed and expressed, and a sample of high purity and homogeneity was crystallized. The crystal structure was determined by molecular replacement and refined to 1.95 Å resolution (FIG. 1A-1B). The overall crystal structure of the SYMRK kinase domain resembles other eukaryotic kinases of the IRAK4/Pelle-type, e.g. BAK1, BIK1. Additionally, SYMRK contains a 32 amino acid long juxtamembrane (JMA) region connecting the transmembrane domain to the kinase core, and a C-terminal tail that is 47 amino acids long.

By mapping the phosphorylatable amino acid residues to the obtained structure, it was possible to separate 20 of them into two regions: the kinase core (S724, S731, S742, S751, S754, T760 and S807) and the C-terminal tail (S877, S885, S889, S893, S903, T905, S906, T907, S910, T911, T914, S916 and S918) (FIG. 1D).

Some of the phosphorylatable amino acid residues at the kinase core have been investigated before; S754 and T760 have previously been identified by (Yoshida et al., Journal of Biological Chemistry (2005) 280:9203-9209), where alanine substitutions of the two residues were tested for kinase activity (Yoshida et al., Journal of Biological Chemistry (2005) 280:9203-9209). When tested on purified protein from E. coli, S754A showed slightly reduced kinase activity and T760A showed no activity (Yoshida et al., Journal of Biological Chemistry (2005) 280:9203-9209). In general, mutations at the core kinase domain of SYMRK have been linked to loss of kinase activity (Yoshida et al., Journal of Biological Chemistry (2005) 280:9203-9209) and thereby loss of symbiotic function in mutants like symrk-7, symrk-10, and symrk-11 (Perry et al., Plant Physiol (2003) 131:866-871, Stracke et al., Nature (2002) 417 (6892): 959-62). The present Example focused on the so-far uninvestigated role of phosphorylation of the C-terminal tail of SYMRK in order to describe the role of SYMRK in the RNS pathway.

### Phosphorylation of C-Terminal Tail of SYMRK is Required for its Symbiotic Function but not for Kinase Activity

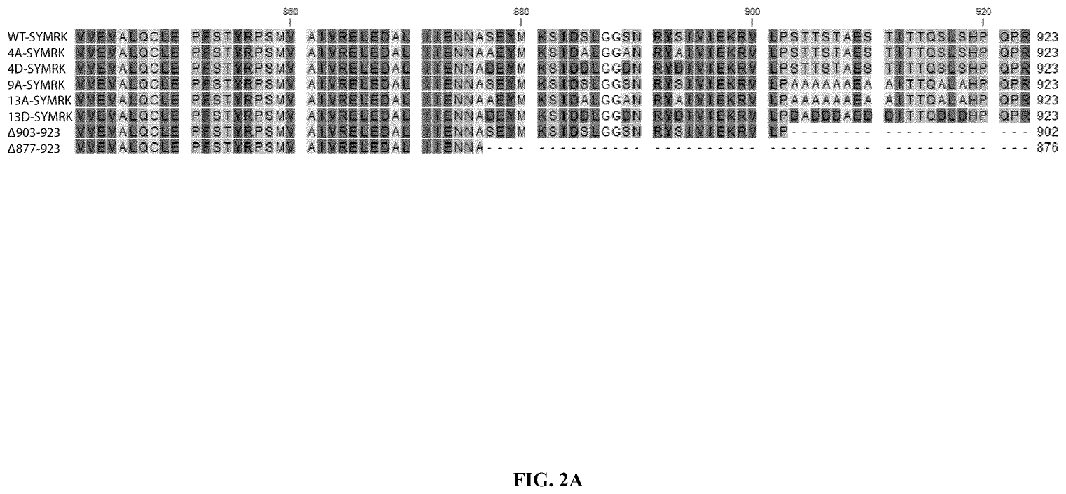

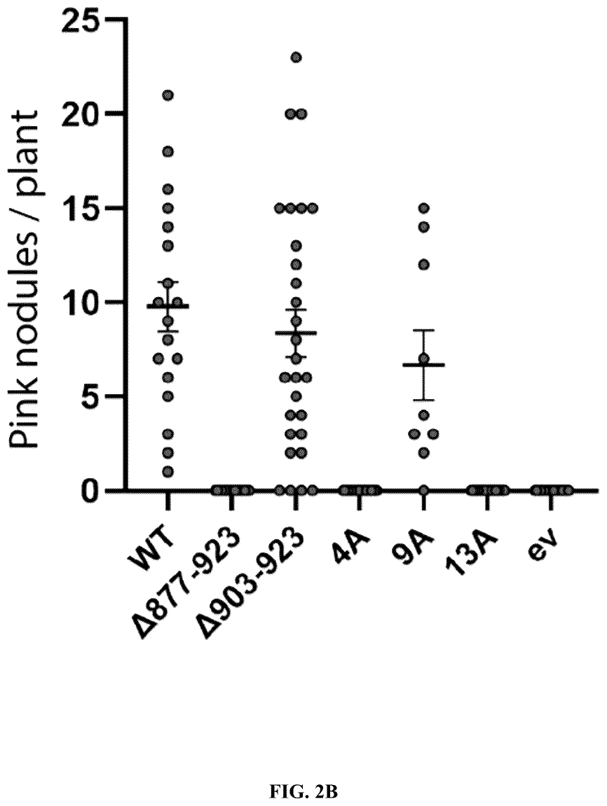

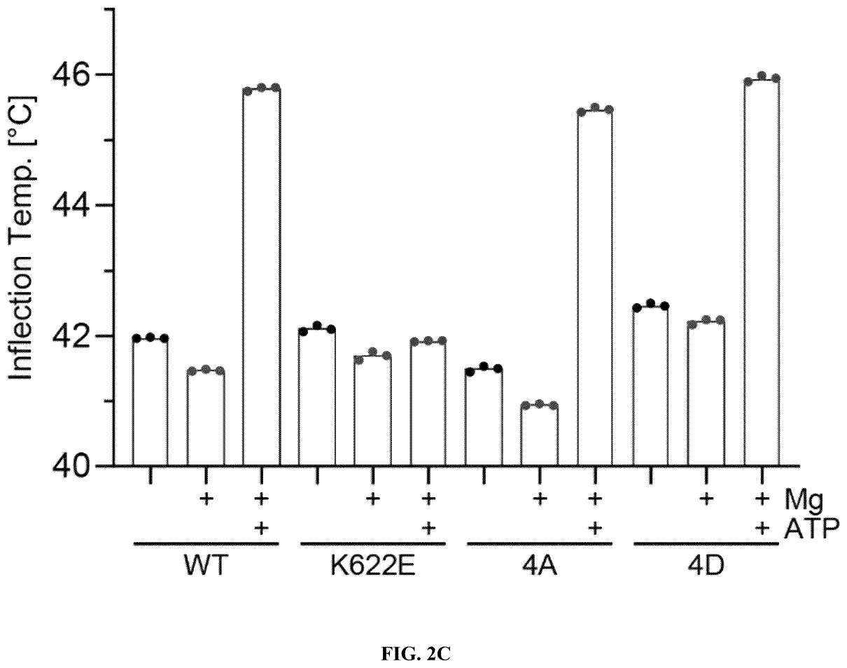

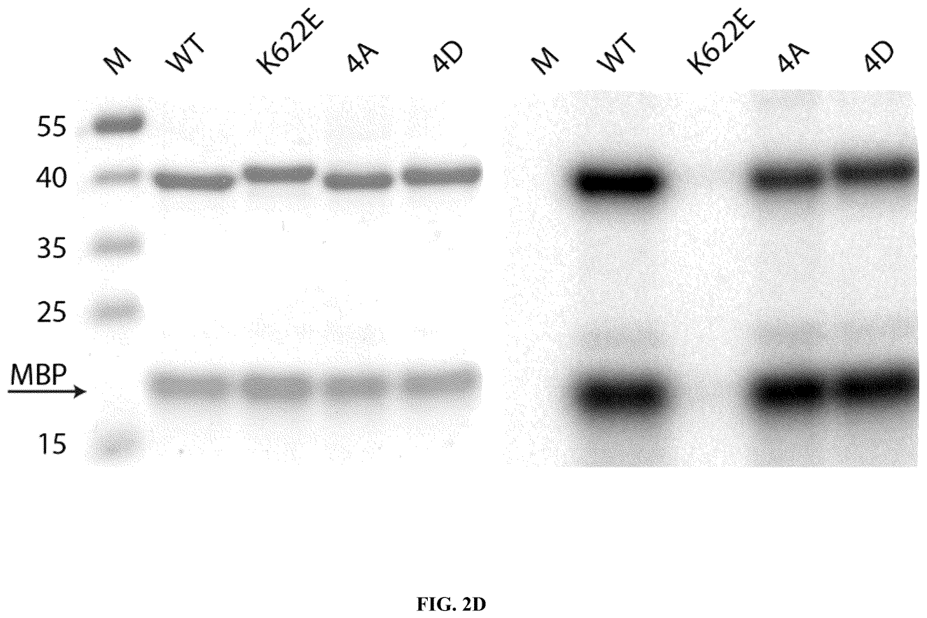

Phosphorylatable amino acid residues in the core kinase have been shown to be essential for kinase activity for e.g., BAK1 (Yan et al., Cell Res (2012) 22:1304-1308), the brassinosteroid receptor BRI1 (Wang et al., Structure (2006) 14:1835-1844), and human interleukin-1 receptor-associated kinase 4 (Cheng et al., Biochem Biophys Res Commun (2006) 352 (3): 609-16) The experiments in this Example focused on phosphorylatable amino acid residues at the C-terminal tail of SYMRK. The C-terminal tail regions of kinases are often phosphorylated (Kovacs et al., Mol Cell Biol (2015) 35:3083-3102), and it has been shown that the BAK1 C-terminal tail regulates its role in immune response (Perraki et al., Nature (2018) 561:248-252). The experiments in this Example identified 13 phosphorylatable amino acid residues at the C-terminal tail of SYMRK via phosphor enrichment followed by MS analysis (FIG. 1D). In order to determine if the sites were biologically relevant for the symbiotic role of SYMRK in root nodule symbiosis, non-phosphorylatable variants for S877, S885, S889, S893, S903, T905, S906, T907, S910, T911, T914, S916, and S918, named 13A (substituting S or T with A) (FIG. 1E, FIG. 2A) were created and expressed in transgenic roots of a null symrk-3 mutant background. The 13A SYMRK could not complement the symrk-3 phenotype (FIG. 2B), indicating that the phosphorylation of the C-terminal tail is important for the role of SYMRK in root nodule symbiosis. To more specifically understand its regulation, a SYMRK lacking its C-terminal tail, named 4877-923 (FIG. 2A), was created. This version was unable to complement the nodulation phenotype of symrk-3 (FIG. 2B); this result underlined the importance of the C-terminal tail of SYMRK for its biological function. These observations are in line with previous studies of other receptor kinases (Perraki et al., Nature (2018) 561:248-252). As the data for BAK1 indicated that most C-terminal sites were regulatory (Perraki et al., Nature (2018) 561:248-252), a SYMRK variant that was non-phosphorylatable at S903, T905, S906, T907, S910, T911, T914, S916, and S918, named 9A (substituting S or T with A), was created (FIG. 2A). 9A SYMRK showed full complementation of the nodulation phenotype of symrk-3 when expressed in transgenic roots (FIG. 2B). This observation indicated that the remaining four phosphorylatable amino acid residues in the C-terminal tail, S877, S885, S889 and S893, are the essential phosphorylatable amino acid residues for symbiosis. S877, S885, S889, and S893 are also the most conserved phosphorylatable amino acid residues in the tail of SYMRK when comparing within both the RNS and AMS clades (FIG. 5). To test this the essentiality of the four phosphorylatable amino acid residues, a phosphorylation-null mutant version was created by substituting S with A (FIG. 1E) at residues S877, S885, S889 and S893 of SYMRK, which was named 4A (FIG. 2A). 4A SYMRK showed no nodule formation when expressed in symrk-3 mutant background (FIG. 2B), showing S877, S885, S889 and S893 to be necessary phosphorylatable amino acid residues for root nodule symbiosis. To test if the most C-terminal part of the tail had the regulatory function of SYMRK, a version of SYMRK with only the beginning of its tail was created, and named A903-923 (FIG. 2A). This A903-923 SYMRK formed nodules like the wild type SYMRK when expressed in a symrk-3 mutant background (FIG. 2B).

The importance of the C-terminal tail for regulating root nodule symbiosis was clearly shown through observations that removing the entire C-terminal tail of SYMRK (4877-923) or substituting the 13 phosphorylatable amino acid residues in it with alanines resulted in no rescue of the nodulation phenotype of symrk-3. The C-terminal sites important for regulation were determined to be S877, S885, S889 and S893 by showing that 9A and A903-923 SYMRK fully complemented the lack of nodulation in symrk-3 plants; in line with this, 4A SYMRK was incapable of complementation (FIG. 2B).

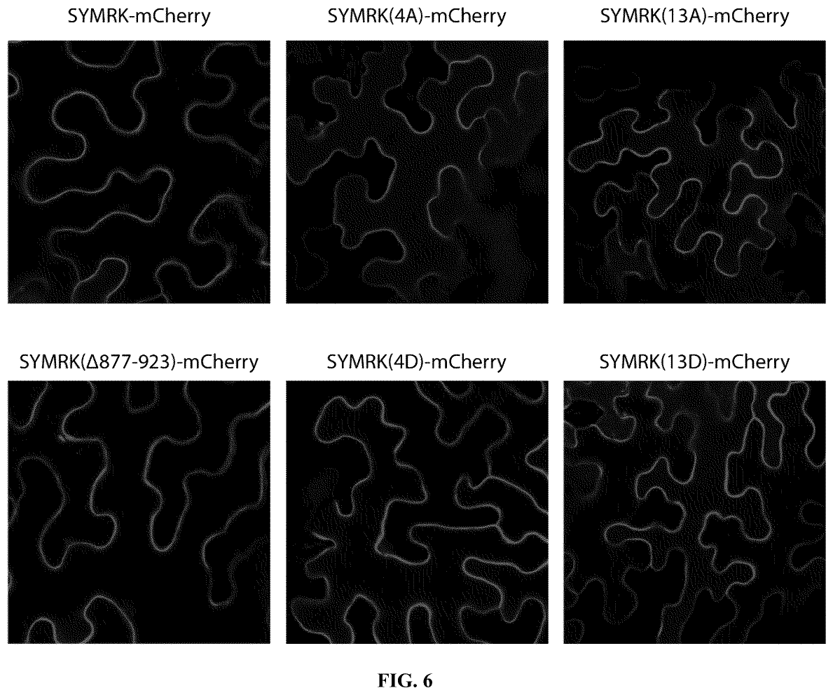

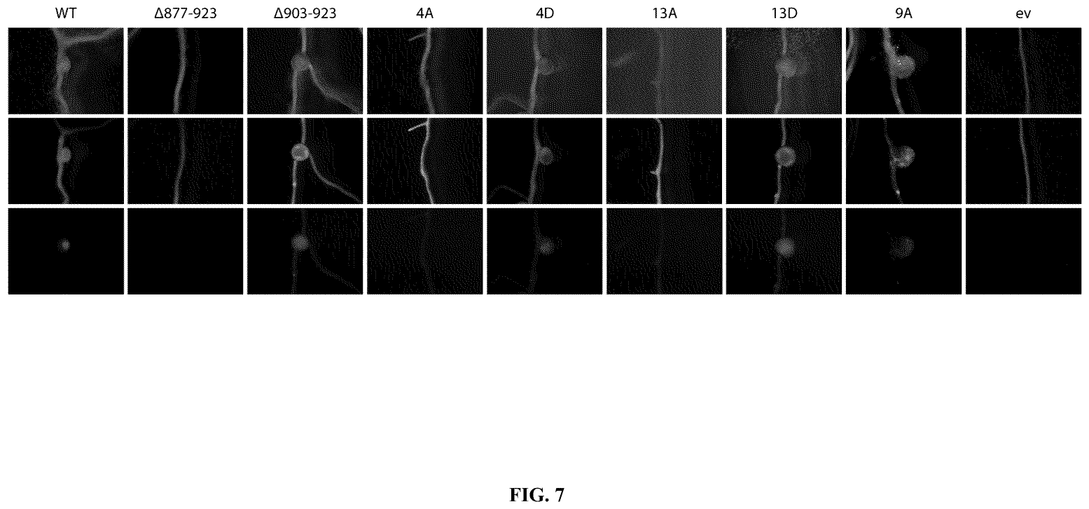

In order to understand if the lack of complementation was the result of an unstable protein after introducing the 4A mutation, kinase domains of SYMRK with 4A mutations were purified. Thermostability of the 4A kinase was similar to that of a wild-type SYMRK, indicating that the folding is similar (FIG. 2C). Additionally, ATP binding was intact in the 4A SYMRK and at a similar level as the wild type (FIG. 2C). A K622E mutant that was previously reported to have lost its kinase activity was used as a kinase-dead control. To investigate if 4A SYMRK retained its kinase activity, kinase domains of 4A SYMRK were incubated with radioactive-labelled ATP and separated on a SDS-PAGE followed by an autoradiogram. The result showed that kinase activity was at a similar level to wild-type SYMRK (FIG. 2D). This indicates that the loss of symbiotic activity seen for 4A SYMRK was not caused by its lack of kinase activity, unlike the first identified null mutants of SYMRK like P55 (Duc 1989), symrk-8 and symrk-10 (Perry et al., Plant Physiol (2003) 131:866-871). In order to determine if the four mutations in the C-terminal tail or the removal of the tail of SYMRK influenced the localization of the protein, it was expressed with an mCherry tag in Nicotiana benthamiana. All tested versions of SYMRK showed similar localization at the plasma membrane as the wild-type SYMRK (FIG. 6). Nodules formed on roots expressing 4903-923 or 9A SYMRK were indistinguishable from the nodules formed on roots expressing the wild-type SYMRK (FIG. 7). The results presented here show that neither kinase activity, ATP binding capacity, nor localization of the SYMRKs tested here were any different than the wild type.

### Phosphor Mimics at the C-Terminal Tail of SYMRK can Activate Organogenesis in the Absence of Rhizobia

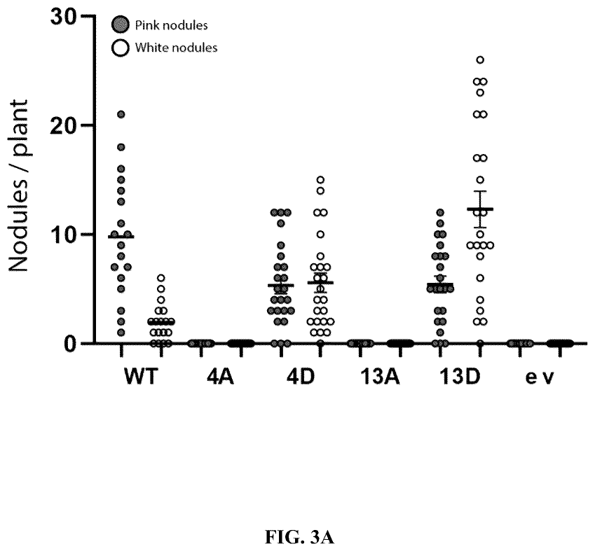

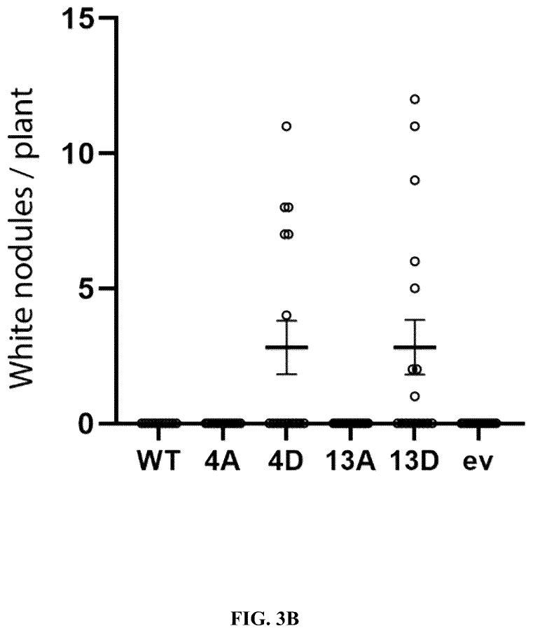

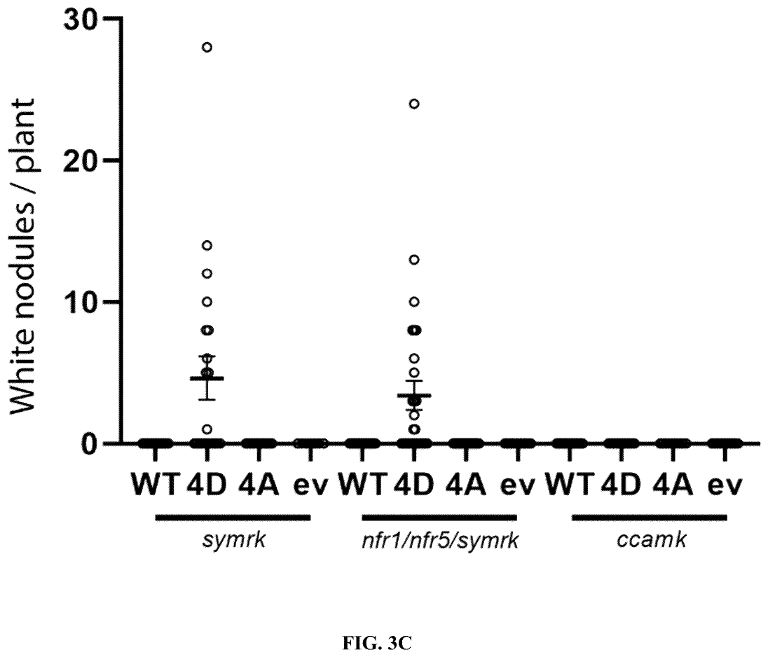

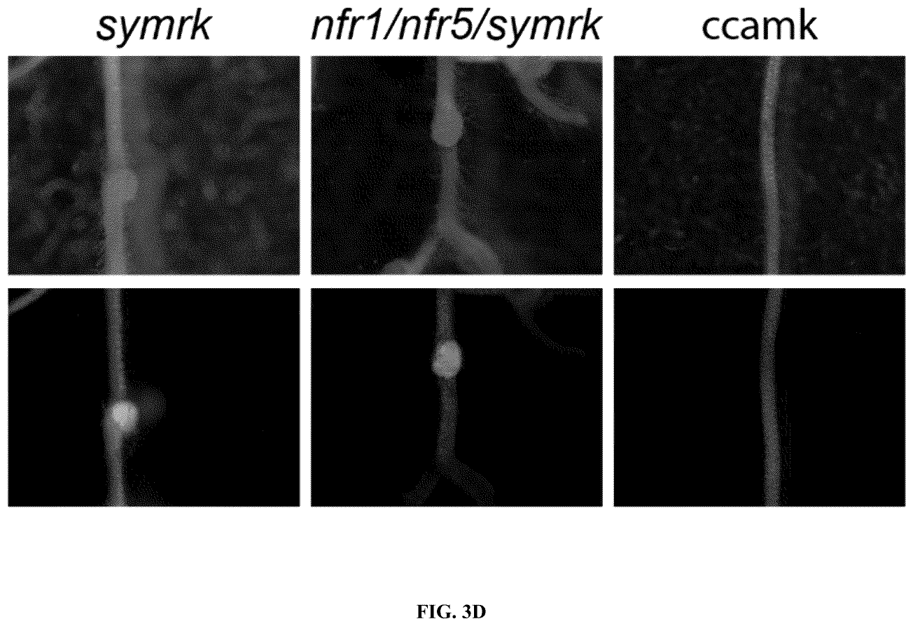

Organogenesis in the absence of symbiotic bacteria has been shown for Lotus plants that have a gain-of-function mutation in the cytokinin receptor LHK1 (Tirichine et al., Science (2007) 315 (5808): 104-7), an auto active calcium calmodulin-dependent kinase CCaMK (Tirichine et al., Nature (2006) 441:1153-1156), overexpression of SYMRK for 40 days in transgenic roots (Ried et al., Elife (2014) 3:1-17), phosphor mimicking CYCLOPS (Singh et al., Cell host Microbe (2014) 15:139-152) or artificial interaction between NFR1 and NFR5 (Rübsam et al., Science (2023) 379 (6629): 272-277). LHK1, CCaMK and CYCLOPS all work downstream of SYMRK, whereas NFR1 and NFR5 are interactors of SYMRK and are believed to work upstream (Oldroyd, Nat Rev Microbiol (2013) 11:252-263, Ried et al., Elife (2014) 3:1-17, Rübsam et al., Science (2023) 379 (6629): 272-277)). Root hair deformation in response to nod factor is observed in symrk mutants but not in NFR1 and NFR5 (Stracke et al., Nature (2002) 417 (6892): 959-62). This, together with the fact that forced interaction between NFR1 and NFR5 only results in organogenesis in the presence of SYMRK (Rübsam et al., Science (2023) 379 (6629): 272-277), indicates that a modification mediated by NFR1 or NFR5 happens to SYMRK in order to transmit the signal further downstream from Nod factor perception. As the present study identified four phosphorylatable amino acid residues on the C-terminal tail of SYMRK as crucial for nodulation, the effects of phosphor mimicking of those sites were then investigated to determine if they could result in organogenesis in the absence of rhizobia. To do so, a phosphor mimic mutant (substituting S with D) versions of SYMRK at S877, S885, S889, and S893 was created, named 4D (FIG. 2A). When expressed in transgenic roots of symrk-3 mutants, the 4D SYMRK driven by the native SYMRK promoter showed pink (infected) nodule formation with rhizobia (FIG. 3A), indicating that the mimicking mutants could rescue nodulation in symrk-3 mutants, unlike the 4A SYMRK (FIG. 2B). The number of infected nodules was reduced in 4D SYMRK compared to wild type (FIG. 3A), but the number of white uninfected nodules was increased (FIG. 3A). Without wishing to be bound by theory, this could be a result of miscoordination between infection and organogenesis in the 4D SYMRK roots. 4D SYMRK was then tested in the absence of rhizobia, and the formation of white uninfected nodules was observed (FIG. 3B). This observation indicated that phosphor mimicking of S877, S885, S889 and S893 was sufficient for activation of organogenesis in the absence of rhizobia. In addition, a phosphor mimic of all 13 identified phosphorylatable amino acid residues in the C-terminal tail of SYMRK named 13D was expressed under the SYMRK promoter (FIG. 2A). Like 4D, 13D SYMRK also showed spontaneous nodule formation in the absence of rhizobia (FIG. 3B). To understand if the spontaneously formed nodules were an effect of dysregulated kinase activity or stability of the 4D SYMRK, the thermostability, the ATP binding ability, and kinase activity were tested, like what was done for the 4A SYMRK. Here, no difference was observed between the 4D SYMRK and the wild type (FIGS. 2C-2D), indicating that like for 4A SYMRK, the phosphor regulation of the C-terminal tail is separated from the regulation of kinase activity. In line with the phosphor ablation SYMRKs tested above, both 4D and 13D SYMRK showed plasma membrane localization when overexpressed in Nicotiana benthamiana (FIG. 6).

### Spontaneous Organogenesis in the Absence of Nod Factor Receptors

Overexpression of SYMRK can drive the formation of nodules in the absence of rhizobia in nfr1 but not in CSP mutants (Ried et al., Elife (2014) 3:1-17). Whether the 4D SYMRK expressed by the native SYMRK promoter could drive organogenesis in a nfr1/nfr5/symrk mutant background was therefore tested. Roots expressing the 4D SYMRK did show organogenesis in the nfr1/nfr5/symrk mutant background in the absence of rhizobia (FIG. 3C). Without wishing to be bound by theory, this result supported the idea that SYMRK works downstream of the nod factor receptors. Spontaneous nodules formed by both 4D and 13D SYMRK looked like previously described spontaneous nodules formed in CCaMK and LHK1 gain for function mutants (Tirichine et al., Nature (2006) 441:1153-1156; Tirichine ct al., Science (2007) 315:104-107), and the nodules formed 40 days after overexpression of SYMRK (Ried et al., Elife (2014) 3:1-17) (FIG. 3D). When expressed in a ccamk mutant background, organogenesis was not observed in the absence of rhizobia (FIG. 3C). This placed SYMRK between the Nod factor receptors and the CCAMK. The results presented here are in line with the observations that overexpression of SYMRK can drive organogenesis in the absence of rhizobia (Ried et al., Elife (2014) 3:1-17). The data presented here indicated that four serines at the C-terminal tail of SYMRK are indeed the regulatory ones for RNS and the result of autophosphorylation. Without wishing to be bound by theory, this could be an explanation for the observed organogenesis observed by overexpression of SYMRK, as a highly increased level of SYMRK could lead to autophosphorylation of the C-terminal tail, ultimately resulting in a subpopulation of SYMRK having the four serines phosphorylated and thereby driving organogenesis.

Phosphorylation of S877, S885, S889, and S893 was observed when analyzing the kinase of SYMRK alone (FIG. 1C), indicating autophosphorylation. Without wishing to be bound by theory, this observation in combination with the observation that overexpressed wild-type SYMRK can drive organogenesis independent of NFR1 suggests no direct phosphorylation of the four residues by NFR1.

SYMRK is involved in both RNS and AMS and is therefore conserved among most land plants (Markmann et al., PloS Biol (2008) 6:0497-0506). At the C-terminal tail of SYMRK, the residues closest to the kinase domain are the most conserved; this includes the S877, S885, S889, and S893 (FIG. 5), which were identified herein to be both necessary and sufficient to drive organogenesis. This points towards some redundancy between SYMRK from different plants inside and outside the RNS family. It has been shown that symrk mutants of Lotus can be complemented by SYMRK from Medicago as well as by non-nodulating species like rice (Markmann et al., PloS Biol (2008) 6:0497-0506)., Li et al., Front Plant Sci (2018) 9). SYMRK from Arabidopsis cannot complement symrk mutants of Lotus (Li et al., Front Plant Sci (2018) 9); Arabidopsis SYMRK, contrary to Lotus, Medicago and rice SYMRK, lacks two out of the four serins identified in this study to be important for nodulation (FIG. 5). The conservation of the four serines in the C-terminal tail, combined with the cross-species complementation of symrk mutants, offers the potential for engineering nodule organogenesis in plants outside the RNS clade, like maize, in order to get closer to engineering nitrogen fixation in cereals.

Taken together, the present study identified four serine residues in the C-terminal of SYMRK as essential for organogenesis. The data presented herein show that those four residues are conserved in SYMRKs outside the RNS clade and, if substituted with aspartic acid, can drive organogenesis in the absence of rhizobia, making them an interesting target for engineering nitrogen-fixing crops.

### Example 2: Five Phosphorylatable Amino Acid Residues in the Activation Zone Negatively Regulate SYMRK Kinase Activity and Symbiotic Function

The following example describes experiments demonstrating that five phosphorylatable amino acid residues in the activation zone negatively regulate SYMRK kinase activity and symbiotic function.

### Materials and Methods

The experiments described in this Example were performed as described in Example 1 unless otherwise noted.

### Results and Discussion

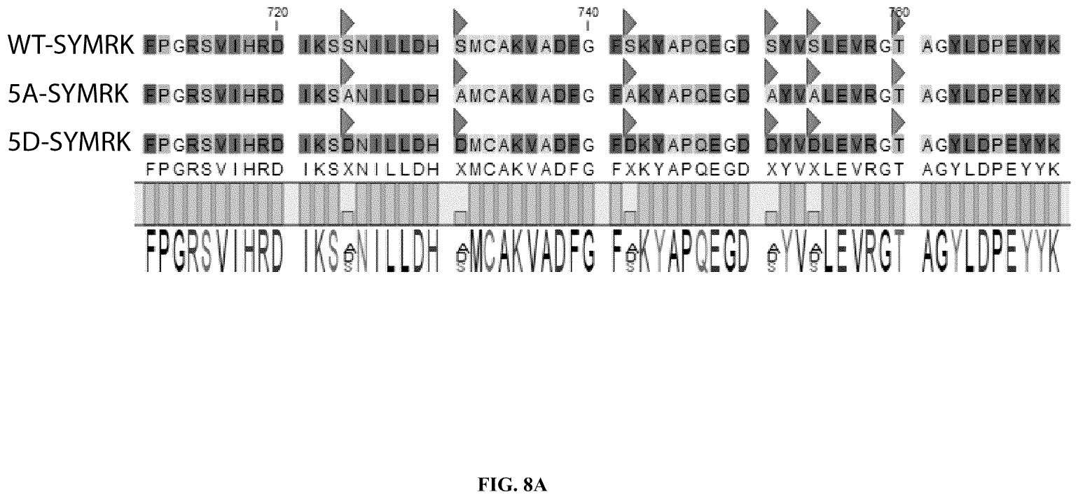

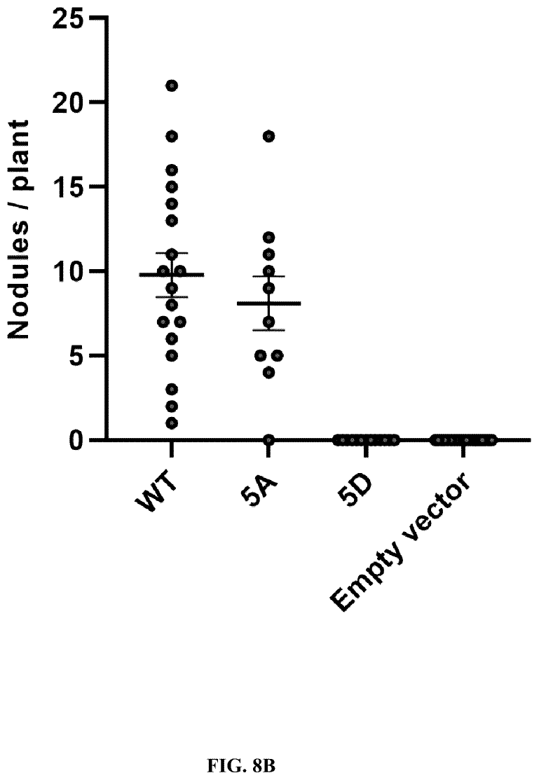

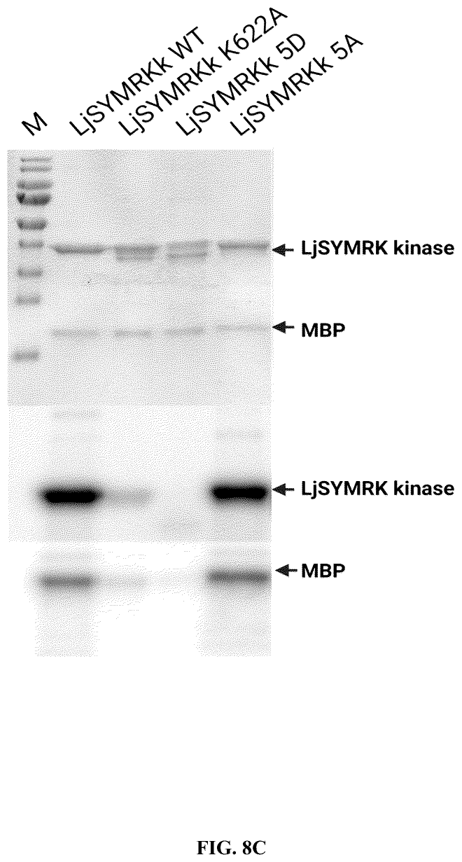

To test the in vivo function of sites identified in Example 1, transgenic roots expressing phosphomimetic (substituting S with D, FIG. 1E) or phosphorylation-null mutants (substituting S with A, FIG. 1E) versions of SYMRK were created and expressed in a null symrk-3 mutant (Stracke et al., Nature (2002) 417 (6892): 959-62) background and tested for complementation of loss of nodulation in response to rhizobia treatment for 4 weeks. For the activation zone, a quintuple alanine mutant (S724A, S731A, S742A, S751A, S754A), hereafter named 5A (FIG. 8A), was tested. T760A was not included in the analysis because it has already been tested and showed no kinase activity (Yoshida et al., Journal of Biological Chemistry (2005) 280:9203-9209). In addition, a quintuple phosphomimetic (S724D, S731D, S742D, S751D, S754D), hereafter named 5D (FIG. 8A) was created and tested in transgenic roots in symrk-3. Surprisingly, 5A showed full complementation of nodulation of the symrk-3 mutant (FIG. 8B). In contrast, 5D showed no complementation of the symrk-3 mutant (FIG. 8B). These results contrast the general observations of the receptor kinases activation zone where phosphorylation of the activation loop is essential for the kinase activity and thereby the biological function. This includes well studied receptor kinases like the immunity receptor kinases FLS2 (Robatzek et al., Genes Dev (2006) 20:537-542), brassinosteroid receptor kinase BRI1 (Wang et al., Structure (2006) 14:1835-1844) and human interleukin-1-receptor-associated kinase 4 (Cheng et al., Biochem Biophys Res Commun (2006) 352 (3): 609-16).

To further investigate the activation zone of SYMRK, kinase domains of SYMRK with 5A or 5D mutations were purified and kinase activity was tested (FIG. 8C). 5A showed kinase activity at a similar level to wild type (FIGS. 8C-8C). This is in line with the nodulation complementation assay where we see wild type like function of 5A (FIG. 8B). Surprisingly, the kinase assay of 5D showed no activity at all and no ATP binding, (FIG. 8C). This fits with the phenotype for nodulation showing no complementation of symrk-3 by 5D (FIG. 8B). Importantly, this links the biological function of SYMRK to its kinase activity in a novel regulation where the activity is negatively regulated by phosphorylation on serine residues at S724, S731, S742, S751, or S754, opposite to the general perception of regulation of kinase activity.

### Example 3: Spontaneous Organogenesis is Kinase Activity Dependent

The following example describes experiments demonstrating spontaneous nodulation is fully dependent on an active kinase of SYMRK.

### Materials and Methods

The experiments described in this Example were performed as described in Example 1 unless otherwise noted. A phosphor mimic mutant, termed 4D, was created by substituting S with D at S877, S885, S889, and S893 of SYMRK. To generate a kinase dead SYMRK, a 622E mutant was created on the 4D mutant background. FIG. 9A shows the crystal structure of SYMRK 4D.

### Results and Discussion

To test the in vivo function of sites identified in Example 1, the 4D mutant version of SYMRK was created and expressed in a null symrk-3 mutant (Stracke et al., Nature (2002) 417 (6892): 959-62) background and tested for complementation of loss of nodulation in response to rhizobia treatment for 4 weeks. A kinase dead version of the 4D mutant was created by adding a glutamic acid mutation at position 622 to the 4D background (hereafter named 622E). The 622E mutant version of SYMRK was also expressed in a null symrk-3 mutant (Stracke et al., Nature (2002) 417 (6892): 959-62) background and tested for complementation of loss of nodulation in response to rhizobia treatment for 4 weeks. Surprisingly, the 622E mutant did not show any nodule formation, (FIG. 9B).

### ADDITIONAL REFERENCES

Antolín-Llovera, M., Ried, M. K., and Parniske, M. (2014). Cleavage of the symbiosis receptor-like kinase ectodomain promotes complex formation with nod factor receptor 5. Current Biology 24:422-427.
Arora, D. et al. (2020). Establishment of proximity-dependent biotinylation approaches in different plant model systems. Plant Cell 32:3388-3407.
Chinchilla, D., Zipfel, C., Robatzek, S., Kemmerling, B., Nürnberger, T., Jones, J. D. G., Felix, G., and Boller, T. (2007). A flagellin-induced complex of the receptor FLS2 and BAK1 initiates plant defense. Nature 448:497-500.
Duc, G. and Messager, A. (1989). THE ISOLATION OF MUTANTS FOR NODULATION AND NITROGEN FIXATION.
Hee Nam, K. and Li, J. (2002). BRI1/BAK1, a Receptor Kinase Pair Mediating Brassinosteroid Signaling.
Kim, S., Zeng, W., Bernard, S., Liao, J., Venkateshwaran, M., Ane, J. M., and Jiang, Y. (2019). Ca2+-regulated Ca2+channels with an RCK gating ring control plant symbiotic associations. Nat Commun 10.
Lévy, J. et al. (2004). A Putative Ca 2+ and Calmodulin-Dependent Protein Kinase Required for Bacterial and Fungal Symbioses. Science (1979) 303:1361-1364.
Murray, J. D., Karas, B. J., Sato, S., Tabata, S., Amyot, L., and Szczyglowski, K. (2007). A cytokinin perception mutant colonized by Rhizobium in the absence of nodule organogenesis. Science (1979) 315:101-104.
Stougaard. J. (1995). Agrobacterium rhizogenes as a Vector for Transforming Higher Plants: Application in Lotus corniculatus Transformation. In Plant Gene Transfer and Expression Protocols (Humana Press: New Jersey), pp. 49-62.

What is claimed is:

1. A modified plant SYMRK polypeptide comprising (i) substitution of one or more, two or more, three or more, or all four phosphorylatable amino acid residues with a phosphomimetic amino acid residue, wherein the one or more, two or more, three or more, or all four phosphorylatable amino acid residues correspond to amino acids S877, S885, S889, or S893 of SEQ ID NO: 2, and/or (ii) substitution of one or more, two or more, three or more, four or more, or all five phosphorylatable amino acid residues with a non-phosphorylatable amino acid residue, wherein the one or more, two or more, three or more, four or more, or all five phosphorylatable amino acid residues correspond to amino acids S724, S731, S742, S751, or S754 of SEQ ID NO: 2.

2. The modified plant SYMRK polypeptide of claim 1, wherein the phosphomimetic amino acid residue is aspartic acid or glutamic acid, preferably aspartic acid.

3. The modified plant SYMRK polypeptide of claim 1, wherein the non-phosphorylatable amino acid residue is alanine or glycine, preferably alanine.

4. The modified plant SYMRK polypeptide of claim 1, wherein the phosphorylatable amino acid residues are serine, tyrosine, or threonine.

5. The modified plant SYMRK polypeptide of claim 1, wherein the plant SYMRK polypeptide comprises a polypeptide with at least 70% identity, at least 80% identity, at least 90% identity, at least 95% identity, or at least 99% identity to a protein selected from the group of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8 or a functional fragment or conserved domain there.

6. The modified plant SYMRK polypeptide of claim 1, wherein the modified plant SYMRK polypeptide constitutively induces symbiotic organogenesis or induces symbiotic organogenesis in the absence of rhizobial bacteria and/or arbuscular mycorrhizal fungi recognized by the plant at a higher level than the unmodified plant SYMRK polypeptide under the same conditions.

7. The modified plant SYMRK polypeptide of claim 6, wherein the organogenesis is nodule formation, arbuscule or vesicle formation, or lateral root formation, preferably nodule formation.

8. The modified plant SYMRK polypeptide of claim 6, wherein the symbiosis is nitrogen-fixing nodulation symbiosis or arbuscular mycorrhizal symbiosis, preferably nitrogen-fixing nodulation symbiosis.

9. The modified plant SYMRK polypeptide of claim 1, wherein the modified plant SYMRK polypeptide comprises an active kinase domain.

10. A genetically modified plant or part thereof comprising the modified plant SYMRK receptor polypeptide of claim 1.

11. The genetically modified plant or part thereof of claim 10, wherein the modified plant SYMRK polypeptide constitutively induces symbiotic organogenesis or induces symbiotic organogenesis in the absence of rhizobial bacteria and/or arbuscular mycorrhizal fungi recognized by the plant at a higher level than the unmodified plant SYMRK polypeptide under the same conditions.

12. The genetically modified plant or part thereof of claim 10, wherein the organogenesis is nodule formation, arbuscule or vesicle formation, or lateral root formation, preferably nodule formation.

13. The genetically modified plant or part thereof of claim 10, wherein the symbiosis is nitrogen-fixing nodulation symbiosis or arbuscular mycorrhizal symbiosis, preferably nitrogen-fixing nodulation symbioses.

14. The genetically modified plant of claim 10, further comprising a genetically modified plant NFR1 LysM receptor polypeptide and/or a genetically modified plant NFR5 LysM receptor polypeptide.

15. A method of producing the genetically modified plant or part thereof of claim 10, comprising introducing a genetic alteration to the plant comprising a first nucleic acid sequence encoding the modified plant SYMRK receptor polypeptide.

16. The method of claim 15, wherein the nucleic acid sequence is operably linked to a promoter, wherein the promoter is a root active promoter, an inducible promoter, a constitutive promoter, or a combination thereof.

17. The method of claim 16, wherein the promoter is a root active promoter, and wherein the promoter is selected from the group consisting of a NFR1 promoter, a NFR5 promoter, a LYK3 promoter, a CERK6 promoter, a NFP promoter, a Lotus japonicus NFR5 promoter (SEQ ID NO: 9), a Lotus japonicus NFR1 promoter (SEQ ID NO: 10), a Lotus japonicus CERK6 promoter (SEQ ID NO: 11), a Medicago truncatula NFP promoter (SEQ ID NO: 12), a Medicago truncatula LYK3 promoter (SEQ ID NO: 13), a maize allothioneine promoter, a chitinase promoter, a maize ZRP2 promoter, a tomato LeExtl promoter, a glutamine synthetase soybean root promoter, a RCC3 promoter, a rice antiquitine promoter, a LRR receptor kinase promoter, and an Arabidopsis pCO2 promoter.

18. The method of claim 16, wherein the promoter is a constitutive promoter, and wherein the promoter is selected from the group consisting of a CaMV35S promoter, a derivative of the CaMV35S promoter, a maize ubiquitin promoter, a trefoil promoter, a vein mosaic cassava virus promoter, and an Arabidopsis UBQ10 promoter.

19. The method of claim 15, wherein the nucleic acid sequence is inserted into the genome of the plant so that the nucleic acid sequence is operably linked to an endogenous promoter, and wherein the endogenous promoter is a root active promoter.

20. The method of claim 15, further comprising introducing one or more genetic alterations to the plant comprising a second nucleic acid sequence encoding a modified plant NFR1 LysM receptor polypeptide and/or a third nucleic acid sequence encoding a modified plant NFR5 LysM receptor polypeptide.

21. A method of producing the genetically modified plant or part thereof of claim 10, comprising genetically modifying the plant or part thereof by transforming the plant or part thereof with one or more gene editing components that target an endogenous nuclear genome sequence encoding an endogenous plant SYMRK receptor polypeptide and introduce (i) the substitution of one or more, two or more, three or more, or all four phosphorylatable amino acid residues with a phosphomimetic amino acid residue, wherein the one or more, two or more, three or more, or all four phosphorylatable amino acid residues correspond to amino acids S877, S885, S889, or S893 of SEQ ID NO: 2, and/or (ii) the substitution of one or more, two or more, three or more, four or more, or all five phosphorylatable amino acid residues with a non-phosphorylatable amino acid residue, wherein the one or more, two or more, three or more, four or more, or all five phosphorylatable amino acid residues correspond to amino acids S724, S731, S742, S751, or S754 of SEQ ID NO: 2.

22. The method of claim 21, wherein the one or more gene editing components comprise a ribonucleoprotein complex that targets the nuclear genome sequence; a vector comprising a TALEN protein encoding sequence, wherein the TALEN protein targets the nuclear genome sequence; a vector comprising a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.

23. The method of claim 21, further comprising genetically modifying the plant or part thereof by transforming the plant or part thereof with one or more gene editing components that target a second endogenous nuclear genome sequence encoding an endogenous plant NFR1 LysM receptor polypeptide for modification and/or that target a third endogenous nuclear genome sequence encoding an endogenous plant NFR5 LysM receptor polypeptide for modification.

24. An expression vector, isolated DNA molecule, or recombinant nucleic acid comprising a nucleic acid sequence encoding the modified plant SYMRK receptor polypeptide of claim 1, optionally operably linked to at least one expression control sequence.

25. The expression vector, isolated DNA molecule, or recombinant nucleic acid of claim 24, wherein the at least one expression control sequence comprises a promoter selected from the group consisting of a root active promoter, a constitutive promoter, and a combination thereof.

26. The expression vector, isolated DNA molecule, or recombinant nucleic acid of claim 25, wherein the promoter is a root active promoter, and wherein the promoter is selected from the group consisting of a NFR1 promoter, a NFR5 promoter, a LYK3 promoter, a CERK6 promoter, a NFP promoter, a Lotus japonicus NFR5 promoter (SEQ ID NO: 9), a Lotus japonicus NFR1 promoter (SEQ ID NO: 10), a Lotus japonicus CERK6 promoter (SEQ ID NO: 11), a Medicago truncatula NFP promoter (SEQ ID NO: 12), a Medicago truncatula LYK3 promoter (SEQ ID NO: 13), a maize allothioneine promoter, a chitinase promoter, a maize ZRP2 promoter, a tomato LeExtl promoter, a glutamine synthetase soybean root promoter, a RCC3 promoter, a rice antiquitine promoter, a LRR receptor kinase promoter, and an Arabidopsis pCO2 promoter.

27. The expression vector, isolated DNA molecule, or recombinant nucleic acid of claim 25, wherein the promoter is constitutive promoter, and wherein the promoter is selected from the group consisting of a CaMV35S promoter, a derivative of the CaMV35S promoter, a maize ubiquitin promoter, a trefoil promoter, a vein mosaic cassava virus promoter, and an Arabidopsis UBQ10 promoter.

28. A bacterial cell or an Agrobacterium cell comprising the expression vector, isolated DNA molecule, or recombinant nucleic acid of claim 24.

29. A genetically modified plant, plant part, plant cell, or seed comprising the expression vector, isolated DNA molecule, or recombinant nucleic acid of claim 24.

30. A composition or kit comprising the expression vector, isolated DNA molecule, or recombinant nucleic acid of claim 24.

31. A method of constitutively inducing symbiotic organogenesis or inducing symbiotic organogenesis in the absence of rhizobial bacteria and/or arbuscular mycorrhizal fungi recognized by the plant comprising introducing a genetic alteration via the expression vector, isolated DNA molecule, or recombinant nucleic acid of claim 24.

32. A method of inducing activity or increasing symbiotic function in a SYMRK polypeptide comprising
(i) substituting one or more, two or more, three or more, or four phosphorylatable amino acid residues with a phosphomimetic amino acid residue, wherein the one or more, two or more, three or more, or four phosphorylatable amino acid residues correspond to amino acids S877, S885, S889, or S893 when aligned to SEQ ID NO: 2, and/or
(ii) substituting one or more, two or more, three or more, four or more, or five phosphorylatable amino acid residues with a non-phosphorylatable amino acid residue, wherein the one or more, two or more, three or more, four or more, or five phosphorylatable amino acid residues correspond to amino acids S724, S731, S742, S751, or S754 when aligned to SEQ ID NO: 2, wherein
the increase in symbiotic function is in comparison to the WT SYMRK in the absence of rhizobial bacteria and/or arbuscular mycorrhizal fungi.


SEQUENCE LISTING (34 sequences)

SEQ ID NO: 1 - protein, 579 aa, Zea mays
MAARSLLLYALLLLALATAAPSHGLTQADVAKRLKEVLSERNRGHEMLDSWNGDGDPCSPSTWEGFSCEPKDGAQVVVKLNFSSKKLQGPIPAEIANLTELNEIHLQYNNFTGFIPASFSAFRHLLKLSVICNPLLNNKQPDGFSSGVNFSYGGCATQEYYSSPAEEYQSPPAVASQKVYVIGGVAGGSLACTVALGSFFVCFNKRERRSPKKDCSSTTNPVFQECSIHNTTNPAVQQLSLKAIQTATSNYKTMIGEGGFGAVYRGALANGQEVAVKVRSSSSTQGTREFNNELRLLSAVWHENLVPLIGYCCEKDQQILVYPFMSNGSLQDRLYGEASKRKVLDWPTRLSVCIGAARGLVYLHNFAGRCIIHRDIKSSNILLDHSMCGKVADFGFSKYAPQEGDSNPSMEVRGTAGYLDPEYYSTQVLSTRSDVFSFGVVLLEIVTGREPLDVKRPRHEWSLVEWAKPYIREYKIEEMVDPGIKGQYCSEAMWRVLEVASVCTEPFSTFRPTMEDVLRELEDALIIENNASEYMRSIESTGTLGSNRYLSIDRKMFASGSARIESMKGNLQAMPSLPR

SEQ ID NO: 2 - protein, 923 aa, Lotus japonicus
MMELPATRILSQAVTCFLCLYIFIGSASATEGFESIACCADLNYTDPLTTLNYTTDYTWFSDKRSCRKIPETELRNRSNENVRLFDIDEGKRCYNLPTIKNGVYLIRGTFPFDSLNSSFNASIGVTQLGAVRSSRLQDLEIEGVFRATKDYIDFCLLKGEVYPFISQLELRPSPEEYLQDFPTSVLKLISRNNLGDTKDDIRFPVDQSDRIWKASSISSSAVPLSSNVSNVDLNANVTPPLTVLQTALTDPERLEFIHTDLETEDYGYRVFLYFLELDRTLQAGQRVFDIYVNSEIKKESFDVLAGGSNYRYDVLDISASGSLNVTLVKASKSEFGPLLNAYEILQVRPWIEETNQTDVGVIQKMREELLLQNSGNRALESWSGDPCILLPWKGIACDGSNGSSVITKLDLSSSNLKGLIPSSIAEMTNLETLNISHNSFDGSVPSFPLSSLLISVDLSYNDLMGKLPESIVKLPHLKSLYFGCNEHMSPEDPANMNSSLINTDYGRCKGKESRFGQVIVIGAITCGSLLITLAFGVLFVCRYRQKLIPWEGFAGKKYPMETNIIFSLPSKDDFFIKSVSIQAFTLEYIEVATERYKTLIGEGGFGSVYRGTLNDGQEVAVKVRSATSTQGTREFDNELNLLSAIQHENLVPLLGYCNESDQQILVYPFMSNGSLQDRLYGEPAKRKILDWPTRLSIALGAARGLAYLHTFPGRSVIHRDIKSSNILLDHSMCAKVADFGFSKYAPQEGDSYVSLEVRGTAGYLDPEYYKTQQLSEKSDVFSFGVVLLEIVSGREPLNIKRPRTEWSLVEWATPYIRGSKVDEIVDPGIKGGYHAEAMWRVVEVALQCLEPFSTYRPSMVAIVRELEDALIIENNASEYMKSIDSLGGSNRYSIVIEKRVLPSTTSTAESTITTQSLSHPQPR

SEQ ID NO: 3 - protein, 903 aa, Solanum lycopersicum
MEVDNCWNIRLVNCVIICLVIYIQSAFAQEGFLSIQCCATANFTEPRTNLSWISDGIWFPENQSCISRPVYKSEHYERARSFSSDISHKWCYSLPTRKEHDYLVRGTFLSVKQEKTLPHSSFVVLIGVTPIATVKSSDELKVEGIFRATRSYTNFCLLKKKGNPYISKVELRPINSDYLKKEPSEILKLVHRVDAGNKAAEIRYPYDQYDRIWRPASNLESQVTQTPPSIIKHVFARKHSLLPPAFVLRTALTHPERLDFLHEDLDTGYYTYSLFLYFLEPNDSVQAGERVFYIYINNEKRLKVDILASGSRYLDVVLNFRANRSVNLTMIKASNLSQLGPICNGYEILKALPRVKETATEEVDIMANVKKELLQQNKNNEIWKSWSGDPCLPLPWPGLTCDRVNGTSVITQIDLSSGGLSGPSPPSIQKLMHLRKLNISINGSSGTNSLFTSYFTYSTRYLSSRIHISNKLSRSIKESNITTDKGMANVKQNSSSTHKLVIGAAVGTALLVILAIVISVVCLFKRRVMAGPKFLMRNYSITRNAVYSVPSMDTTMMKSISSRNFKLEYIEAITQNYKTLIGEGGFGSVYRGTLPDGVEVAVKVRSATSTQGIREFNNELNLLSAITHENLVPLIGYCCENEQQILVYPFMSNSSLQDRLYGGAAKRKILDWPARLSIALGAARGLLYLHTFSERCLIHRDVKSSNILLDQSMCAKVADFGFSKYASQEGDSGTSLEVRGTAGYLDPEYYSTQRLSAKSDVFSFGVVLLEILTGREPLNINKPRNEWSLVEWAKPLIRSSRVEEIVDPTIKGGYHGEALWRVVEVALACTETYSTYRPCMADIVRELEDALIIENNASEYLKSLDSFGGSNRFSVERSIVLPPIKSQTEPSSLLSKPAPPQPR

SEQ ID NO: 4 - protein, 925 aa, Medicago truncatula
MMELQVIRIFRLVVAFVLCLCIFIRSASSATKGFESIACCADSNYTDPKTTLTYTTDHIWFSDKRSCRQIPEILFSHRSNKNVRKFEIYEGKRCYNLPTVKDQVYLIRGIFPFDSLNSSFYVSIGVTELGELRSSRLEDLEIEGVFRATKDYIDFCLLKEDVNPFISQIELRPLPEEYLHGFGTSVLKLISRNNLGDTNDDIRFPDDQNDRIWKRKETSTPTSALPLSFNVSNVDLKDSVTPPLQVLQTALTHPERLEFVHDGLETDDYEYSVFLHFLELNGTVRAGQRVFDIYLNNEIKKEKFDVLAGGSKNSYTALNISANGSLNITLVKASGSEFGPLLNAYEILQARSWIEETNQKDLEVIQKMREELLLHNQENEALESWSGDPCMIFPWKGITCDDSTGSSIITKLDLSSNNLKGAIPSIVTKMTNLQILNLSHNQFDMLFPSFPPSSLLISLDLSYNDLSGWLPESIISLPHLKSLYFGCNPSMSDEDTTKLNSSLINTDYGRCKAKKPKFGQVFVIGAITSGSLLITLAVGILFFCRYRHKSITLEGFGKTYPMATNIIFSLPSKDDFFIKSVSVKPFTLEYIEQATEQYKTLIGEGGFGSVYRGTLDDGQEVAVKVRSSTSTQGTREFDNELNLLSAIQHENLVPLLGYCNEYDQQILVYPFMSNGSLLDRLYGEASKRKILDWPTRLSIALGAARGLAYLHTFPGRSVIHRDVKSSNILLDQSMCAKVADFGFSKYAPQEGDSYVSLEVRGTAGYLDPEYYKTQQLSEKSDVFSFGVVLLEIVSGREPLNIKRPRIEWSLVEWAKPYIRASKVDEIVDPGIKGGYHAEALWRVVEVALQCLEPYSTYRPCMVDIVRELEDALIIENNASEYMKSIDSLGGSNRYSIVMDKRALPSTTSTAESTITTQTLSHPQPR

SEQ ID NO: 5 - protein, 930 aa, Manihot esculenta
MMGIHNWIVRGVECFIACLLIIIPLTCAEEGLNIKCCAASNSTDIRKLTWISDYGWFPDDASCQPITRAEANFTGYDVRFFKIDSGKRCYKLPTNKDQDYLIRGTFLYGDSVGSLDSFDVLIGVTKISRVNTSEDLKVEGIFRAKDQHTDFCLVHDKGYPYISQLELKPLEDSEYLNVLGKAPNILKLVSRNDVGNSGEDIRYPDDPFDRIWDTDSKLVKPSPDLTLARSYIHNASTTVPIRVLQTALTHPDRLEFLHSSLDAEYHNYTLFLYFFELNSTIKPQQRVFKIYINNEIHQDEFDIQANGSYYNEVALNVTAKEALNLTLVKVSNRSVFGPILNAYEILQMYSWIQGTNQQDVDTIEMARYELLQHNKENQVLQSWSGDPCHPLPWKGLTCQSMNGDPVITRLDISSSRLRGPLPANITRLNNLMQLNVTHNKFTARIPEFPSSSKLTSVDLSHNELVGFLPNSLTSLPNLTTLYYGCNNLSKGTPSFNNSRLATDYGVCVNVTQSSTRNIQGILIGTVACGSFLLALGVALVCIYRQKFIAQRNFDGKRHPMTKNAIFSLPSSDDVTFKSITIQMFTLEYIENATHKYKTLIGEGGFGSVYRGTLSDGQEVAVKVRSSTSTQGTREFENELNLLSSIRHENLVPLLGYCCENDQQILVYPFMSNGSLQDRLYGEAAKRKTLDWPTRLSIALGAARGLTHLHTFAGRCVIHRDVKSSNILLDQSMNAKVADFGFSKYAPQEGDSGASLEVRGTAGYLDPEYYSTQHLSAKSDVFSFGVVLLEIVSGREPLNIHRPRNEWSLVEWAKPYIRESKIDEIVDPSIKGVYHAEAMWRVVEAALACIEPFSAYRPCMADIVRELEDALIIENNASEYMKSIDSIGGYSLGGSNRFSIVIDKKNVIPPTPTPTEPSPINAQAMAPPEPR

SEQ ID NO: 6 - protein, 576 aa, Oryza sativa
MAARFGPLLLVLPLSSALLVLSAATAPRGRPSQVDVGRQLREELWERNQGHEMLRSWRDGDPCSPSPWEGFSCRWKDGNLFVVKLNFSSKKLQGPIPAAIGNLTELDEIDLQDNNFTGSIPESFFDLTHLLKLSVKCNPFLNNQLPHGLSISVEFSYGGCAYHSPPGASNQRIAVIGGVAGGSLACTFALGFFFVCFNKREKNPQKKDCSSTRNPVFEECSTHKATNSAVQQLSLKSIQNATCNFKTLIGEGGFGSVYRGTLAHGEEVAVKVRSTSSTQGTREFNNELRLLSAVRHDNLVPLIGYCCEKDQEILVYPFMSNGSLQDRLYGEASKRKVLDWPTRLSVCIGAARGLAHLHGFAGRCIIHRDVKSSNILLDHSMCGKVADFGFSKYAPQEGDSNASMEVRGTAGYLDPEYYSTQSLSTKSDVFSFGVVLLEIVTGREPLDVQRPRDEWSLVEWAKPYIREYRIEEIVDPGIKGQYCSEAMWRVLEVASACTEPFSTFRPSMEDVVRELEDALIIENNASEYMRSIESTGTLGSNRYLSIDRKMFASGSARFASFDATKGHLQTMPSLPG

SEQ ID NO: 7 - protein, 563 aa, Hordeum vulgare
MVAPFPLLLLVFSAQLLITSAAAASPHGLTSQVDVAKRLKEELWETNPGHEMLKSWNGVDPCSPSAWEGFSCRLLDDNQLVAKLNFSSKELQGPIPAAIGNLTELTEMSVKCNPFLSNQLPDGLSANLTFSHGGCAAEEYSTPPGAANQRTFVVSGLAGGSLACTFALGLFFTCFNKRERRSQKSDCASTTNPVFEERSIHITTNPAVQQLSYKSIQSATNHFKKLIGGGGFGAVYQGTLAHGQQVAVKVRSPASTQGTREFNNELRLLSTVWHDNLVPLIGYCCEKDQQILVYPLMSNGSLQDRLYGEASKRKVLDWPTRISVCIGAARGLVYLHNFAGRCIIHRDVKSSNILLDHSMCGKVADFGFSKYAPQEGDSIASMEVRGTAGYLDPEYYSTQVLSTKSDVFSFGVVLLEIVTGREPLDVQRPRSEWSLVEWAKPYIRDFRIEEMVDPGIKGQYCSEAMWRVLEVATACTESFSTFRPSMEDIARELEDALIIENNASEYMRSMESTGTFGSNRYLSIDRKMFASGSARMDPAKFASGSGRIDAGKGPLQAMPSLPR

SEQ ID NO: 8 - protein, 929 aa, Arabidopsis thaliana
MGLCLAQLAVTCLFLVPFVLSQVTEFVSIDCGCSSNYTDPRTGLGWVSDSEIIKQGKPVTLANTNWNSMQYRRRRDFPTDNKKYCYRLSTKERRRYIVRTTFLYGGLGSEEAYPKFQLYLDATKWATVTIQEVSRVYVEELIVRATSSYVDVCVCCAITGSPFMSTLELRPLNLSMYATDYEDNFFLKVAARVNFGAPNMDALRYPDDPYDRIWESDINKRPNYLVGVAPGTTRINTSKTINTLTREYPPMKVMQTAVVGTQGLISYRLNLEDFPANARAYAYFAEIEELGANETRKFKLVQPYFPDYSNAVVNIAENANGSYTLYEPSYMNVTLDFVLTFSFGKTKDSTQGPLLNAIEISKYLPISVKTDRSDVSVLDAIRSMSPDSDWASEGGDPCIPVLWSWVNCSSTSPPRVTKIALSRKNLRGEIPPGINYMEALTELWLDDNELTGTLPDMSKLVNLKIMHLENNQLSGSLPPYLAHLPNLQELSIENNSFKGKIPSALLKGKVLFKYNNNPELQNEAQRKHFWQILGISIAAVAILLLLVGGSLVLLCALRKTKRADKGDSTETKKKGLVAYSAVRGGHLLDEGVAYFISLPVLEEATDNFSKKVGRGSFGSVYYGRMKDGKEVAVKITADPSSHLNRQFVTEVALLSRIHHRNLVPLIGYCEEADRRILVYEYMHNGSLGDHLHGSSDYKPLDWLTRLQIAQDAAKGLEYLHTGCNPSIIHRDVKSSNILLDINMRAKVSDFGLSRQTEEDLTHVSSVAKGTVGYLDPEYYASQQLTEKSDVYSFGVVLFELLSGKKPVSAEDFGPELNIVHWARSLIRKGDVCGIIDPCIASNVKIESVWRVAEVANQCVEQRGHNRPRMQEVIVAIQDAIRIERGNENGLKSSSSSSSKAQSSRKTLLTSFLELESPDISRNSLAPAAR

SEQ ID NO: 9 - DNA, 1317 bp, Lotus japonicus
  source 1..1317: mol_type = unassigned DNA
ggacatgagattgaagctccaaaattagctcttttttctgatgaatacttaatgctttgttgtattcacttgattaagtgctagaaatcatctttgcatgatcatagattaaatgaatttccagttggtgtgtggagagctattttgttatgctgacatctgcaatttgcagggcatctaatgattgtcatttcttaaattattattggttgtttccgtttctttaattatctgttttaatcttgcaggtcatacaaattaaaatactagccaccacccaagacatactaaatggggtagtagagggaagggtaaggtcgataaggatgactttttattctataaaatttaggagaatttgagcttaagtggcaaggcaaacgacattactatacgaattggctttgtaccagaaacagggaacaaataatattttacaaataagctattatcatgtcagctcatttgttcaactttgatttgattaaaaattaaatgaagttgaatttgttgagctgctttattatatatgccactggatgtttccgcattctaagtgcatgtttgaaaacatttctacaattgattacgaaggaaaaattaatcatggagagaagcttatgtgcgtagcttctgtatttctgaattgattctatctgtacagtagcatttagataatgaatgatcttggttctcgctaagcatcaaaccaatctctacccttttaaaattgcaagaattataagtcatgcattgacccaaatccttctgtggttatgccccttaaaaatccggcaagacatcaagttagttggtcattagggttccaccagctagctgacaccttgtacaacaactggccgtcctaaagttgggtaagcattacaatactaaatgccattttattatattttgcgcatggttatatacctaagtaggatttgtccacagtttctttgattcggaaaggaaaaaatatttagttgacactgacagaagcagattttatatacatatattatgaaatgactcctacatgagatacacgaatctcatccccatgagttgcagtttgacagagtacacacttatcaacttgctggaatataggaaagtctaaccaatgatgtcgatccgtattgccttaattttggtaaatttagtattacatgatcattattgatatactaaaccacaggatattttattgacaatgtgaatgttccatattttcaacaatgctgattccctctgataaagaacaagttccttttctctttccctgttaactatcatttgttccccacttcacaaaca

SEQ ID NO: 10 - DNA, 4171 bp, Lotus japonicus
  source 1..4171: mol_type = unassigned DNA
gcatgcatatagctctatttctttagtaatgttacacctgcacgatgtccataataatagaagtcataatacatatacagattaaaattaaataaacaatttctaatcaaatttaaaaatgtcaacttaatttcattattaaaatataacaatatgaataaccaaaaataaattaagacattcacccccccccccccgaaaagaaatttaagacaattacaattttttggtatatatattaaagacttccaattatggacataggatctcaacttagtaatcttcactttaggaaagtcttttccccacaagtcacaaccatctattaatatcaatacaaaatgaagtcaactcaataaaaagatccttttataggaaattgatgaataaaactgatatatatttcagttaaaattgttcaaacattagtgcaatggacagaagtatcctttgtgccctcatttgccaacaactggctcatcaagcaataaattaattcgccatttccaaacttttgcagttttaagtagaagatatccattcgttgaaactttcttcacaccaccaatttcctcctaaatgggttaacaaatgtgcaatgatcgaaatatatagttgaaacgatcaagatcctctcaatggtaaaagaatttgaccacgctaagttttattatctcactagctattaatttaattatcatttatcttttcaattattaaacacacaaataatcaatcctaaaatgatgaaacttgacatggtctatttttacaataacttaaccaaaaacttataagttagcaactttcaaaaacaggttttcccttgttaagaatagacaaatcaaatggagtgtgttaaatattgtgtttaaaatagtgtgttgcaagcatttctcttataaaaaatcagtataaatatgtttggaactgtttattttagtttatcttatattataaatacaaaacaagtgtttggtaaagctaatgaaaataacttaaaacatacctatggtttgtcggatgtgtcgcgggtggagctccttgccattttgtgtggcctttgtatgtgttgagatatggccatgggttattgtagaatccttcttcttttggattcttctttgttcctgtctctcattcaacgctcatgttacccatttcatccatgccaagttttttttatacatgcatctaaattttttccgccatatcttaattttgtttttattaaaattaaaaagaatatgattgaatgtcaatgtaaattttttttacacagacaatgcatatccattaaggtttgttagaattacactccaccccatttttatctaaaatctacatcccaccccattttatatagaggcaaatttagtgacgaaaaatattcttcattaataattagttattatttaaattgttaatcaataatttcaaaaaaaaattcaatataatccaataaatttaaaaatgaaaacatcacaatcctcctcattctctcaatcgcgttttacctccgtaaatttacaatgcaaattatgcaatagcacacctgcccgatttacaaaccatatttcgaacatagtgaaacatgcttgtgttttcatatttggtgataattcaattttaatcaaaataatctctttatacctccaattttcaaaattgggttgtaggccaaaaaagcaacacaaatgggtgaagaaaatagagaaacaaaattatgaaaatatgaagtggatctgaggttattagagcccaacgaggcggtggagcatcgtttttaacaaaatccaacaatatctttaggggtgaaatccaccaaccgagcgttcgctcacaacttaaaggggtgaaattcacaagaagtagtttaaaggggtgaaattcacaagaagtagttgaacaagtgactttaggaatgtgcgattcacgttctggggttcaggtcgcaacaaaactttgaggcatggtggtgccatgtggtttcacattgtcggacagtggagccgtgttaaagggagtaaaggcttggtggtggccgtttgtggtgaaaaatatgatttggagatagatgggacgtggacttaacagacagagtggatggtttttttttaaatttaattcagtaaaattatttttataaattaatgtatgatagtgtatatgcattaaatttatttaaatttttactaattagtaatttgtttttagtagtgacgaatttgtttttgtcactaaattttgcctttataaaaaatggggtggagtgtagatttttaaaaagaatggggtggagtgtcattcttgcaaatcttgaggggggtgagtgtattttactcaactcttaaaaaaattaggaattaattagttgtaaattataaaagtttatttcattgaataacataacaaattaaaggcaaaaaaatacaaaacttcattttatatgtatttcagaaaaattgcctactttcaattatgagaaactaaaattatgtttagtttaaaatgagcatagattcaaaaattaataaataatatatatagcaggatacatgcctatcaattaacatatcgtttgtccacgatgatgatcttattggaggatcaatatcttcaaattaacaaagttatcacttggctcttattggtcataatgcaataaaaaaattgcaattagtatcaaatcaaactgaaatttgcaactatatgctgctggtgttgtcgcgcagattcctttttgatttttatgggaatgaagtcaatgaagcaacagtttcacaggcgtgcttaaaaataaaaaaattggaaatttgatgtttgttaggattatgagaggacacaatgggaggatgtttcacaagctgcagacagggttgccacttcagatgcaaaggattaaataaacaaagccaaggtttgcaatcaacaagattccatcgtcgttttgcttcctttaatcgtattaatcaaaagcacaccaagtaaagcatcaatatataacatccaagaaatcacaacatgatagttgctcgtgtcgtctattaactatgatgtcaggagttcgatccccgctcatgtgaatggaagtcatttcgttgttagatgtttaccgtttaatgcaaatactcgcggtgagataataagtcattgttgtgggcgaataccctaaaataagaataaaattaaatatagcatccaagttattgcccaaatatataaacaatggtattgttgacattattaggcataaaagcagtaggtaagtgtattatatttatttaattttttaaaattttgaaattaattaataattgttaacataagtaaaccatttttagcaaaaactctacacttctattaccttaacaagtacatttttgatggtacaccttaacaattaacaagtcatatgattgacaaacatattttatatgctttacaatttattctaaaatcaaagtttatgggaagaagctcataaaagtagttcctgggtgttttttagaatagagaagttgatcatgttagaaattaagttaaaaatgagttgaaagtgatttatgtttgattatatttatgagaaaaatgaattgtctgatgtaatattgtaaaatctaacaattaattaagtaccacagaaactagaatttatagcttcaccttagaattgattttggagttaaaatcaattattaaaggagcaattattaaaggagacatccaaatacactagttaattttgacaatcaattctaacacttgcaaatgtgtaaccaaacttactatcagtaagtgaactaatgattcccaagtcaacttttgttctagctagccaaccgttactatgttccctccacaatacattctccttgaaactgtcaagtgtcaactgcacccaaacatccttgtttgtgatgaaaagatcgaaaacgtgtgcttatgaatttacatgtttacattcaccaaaaatcaaaagttacacctctatacttatcacatatgtttgagtcactttccatataaaatcccatagtctattaattatcagagtaagtgtgacattagtttcaagagaaaaataaatgatcaaaacctggtagagagtcctagaaattcaatgttctgatttctttcattcatctctgctgccattttgatttgcaca

SEQ ID NO: 11 - DNA, 2496 bp, Lotus japonicus
  source 1..2496: mol_type = unassigned DNA
tcgactatataaggggttcctccatacctaataagacatgtttgtttcatgtcgatctacttactctattttatggttttctcgtgagcaatttacatataaaatacacattaaattgcacccaataactaactagttacaagattagagcacataaatcatggaatgaaacaatagcctatgacttgggtattcggtccacaacatctctagaagttcgtacaacaacgatcaaagagaaagtactgactagtgatatgagaaatttggtcaggaacaatgggctggacacattacgaacttgagcctgaatagtagcagcatatgtctgccttcgcaatgacgaagaaggttggacggagctaaagttaaagctggaagggtgtgtcgtggcatttgaaatttagggttcaactgtagctttgtttgtctaactacccttctagctttaataaaaaaacgtaaatcaattaattaaagagaaatcacgccaacctgtctcaaacccgctctaacttgttggttaaggttatgtgttgtatttcaatttgcttgttgcttcaacttgactccttacctcctttcatgttttttcttgaaaattgtgtaaactcagttttttttttttgagaatgtatgagaatgtgatacaaattaaaatacaaacatacattgaatgagcggggctaaaatgactcaaggtacatatgagttaagtttttaccaacccatctctttgatgagttgggtttaattgggttagctcacgtgttaactatttcgacaattctaactttttggaataatgccatccaaccctaggtaaaactcaacatgacttctcttaaagaaatgaagagtgaaagtctacataaatcttaaacacttaaaacactacaacttggtagatcattaaaatatgctccagaaataccataatacttgaaaataaataatttataaatgtaaaatataaatttgttgctgcacccaagtctgagtgaggaatgaataaagataaataaaatatatatatgcgttgatactaacatatgatatgatgtgatagatgtcatatataatgaacagaatgatagaaattgttgagcgcatggaagcttcttgttcaataattcaagagctgcttgtggagggctgctgcagggttgttatggatgcaattatcaaatatcttgctggaaattgatccaaaagttgactattaccttatcaatgtccggtgtccttcaagccaccattcaattagtcaataaccttttgtttttgtttttttataattttggtgccaaataattttaaccatttatttttatcatatatattatataagatggcaagtctataatcatttaaaattaaattagtctaaatttttctaatatgctgtataaatttattatatataagaataaataatattaaaaatattgtttaattgctaccagttcaatcattaattaactattgactgaaaagaaaaatgtctaacaaattttgttacattctgtttgttttgagaaaaaaaaaattgtttttcattttctgaaaatatttatttttaaatttattaaatttagaaattatgtttgtttttacgctttgttttcatcttcaccgtcaccaccatcgctaccactgtcgtcactattactatataccactaccatcgttgttgccgttgtcattgtcaacaccattgctgtcgtcgttgttattatcgccccacaaccactatcatcatcactgctatcgtcatagctaccacgaacaccaccatcacttttcattgaaaatgaaaaatgtttatttatttattttgattatattttaaaaaatatttttaatcaaccaaatttctttttaactttttttttcttatgaaaaaaaataatatgaaaagaacaaaccaaacagatccttatattcttgtttccattctttatcacccatgagagtagctgtagcgcgttaactttctaatagaggcgggaatttaccgttaaccgttgggtcatcactcagcttactgagtcatcttcttcctactgtaggcggaacccacacgccactgcctctctattattacatcaaattctattttttttacatcatcatcaagataaataaattgcattattcagaaattaattcctgaatctccctatcttaactcaggtgtctctagctattaccacttaagctattattagaagtctacgtcaaattctataataaacacatattcttattcttattattatactaatacaaaccttgatttgaagccaaaaatgataaaacaattgcagactcgttcaaacaacagacaacatgtaccattaaattccaccccaaattctcaaagcaaattctgaattctccaactagtttccattctcctagtcaactcaactcaaaacccagatgagaatgagataattccaactttaacaaacacacacaattcatcattca

SEQ ID NO: 12 - DNA, 1135 bp, Medicago truncatula
  source 1..1135: mol_type = unassigned DNA
cgatccttcgttgtattcacttggtcaagtgcaagaaatcatctttacacaatcatataatgaatggacctttggttgatgtgttgagagctacattcagtcctctgctgacttctgcaacttgaggggaatctaatgattgtcttattgcaattaacaattccttttaattttttgacagaaagagttggttacaaaatctcatctaacatattgttgagttcctcttttttttttttttttttttttttgataaaagggtacatctgttcgcagctctgaagattaatccctcgaatagggttggataacatgaacgacaaatttgtttcttaagagttttaacactgtcatattcttatggcagaattcgaactcgaacccactggttaagctagaagtaagaagtgacccatgccatctcattcaatcgttcttggcttgttttcggtttagatgaatatgaagtacaaaaagctacgctcaagcaaaggagagcaggtaagaagggcatcttatttttttaatatattagatgtcaagcttacgtggcgatgtaaacactgtttctatccgaattagctatgtactagaaatagggaacaaataatataaatggtgagtttgtcaaggtgttttaatatattgaaaccaatgtaactggaagtgtcaacattgaaagctgttcattgcctgtattatattataattgcatatatataatggcattagcctttgttaaacgttaaaccatatcttttgctagtttgccccttaaaaatttggtaaaggcatcaagttagatggtctttaggtaccagccagctagttgacatcgtgtatggacaccaaattgggttataataccatattattcttaccatctttattatattctgcgcatgatttcatacttgtttgggatttgccaacagtgtctaagatttgaaaaagaaaaatagtagaactaatgacagagacatcagcatatatttttaatatcaaaccaaaagatatttgatgtccaaagaagagataaatataaagttagaagtatgacaataagtcttggttgttatttgccataagaaactctcttttctcttcccctcataatttgcatttcctcacaaca

SEQ ID NO: 13 - DNA, 2000 bp, Medicago truncatula
  source 1..2000: mol_type = unassigned DNA
aaataatgtgtctaaaattaacaaagatattttatggttaatttattgagcttagttaaagtcattttctaaaacgagacattgtagttgttagataaataatttcctttaattttcatcaaagtttgtaaataattatagggcaggacacatatgtccatcaattgatatatatatatcttttgtaataacgatcttatagaagaatcaatatctacaaagaaagttatcacttgtctcaaattatgctttatgcaactggtattgtcatgtagattccattttggtttatatgatattgaagtcaacgaatcacagctgcttaggcgtggttcataatttaaaattgaaactatggtgtttgttggaattatgagaggacagactgtgaagtggttgcacagctaccgacagggttgccattactgatgcaaaggattaaacaatagaacattttcaatcaacttaatctatgtttgacatcacgctcgatgtgtcagaatcacgacgacccactgtaatttttaaaaaaattacattttataatttttgtagaatcacggtgtatcacagtgattttgacatacccactgtaataccaaacatgcacttgcattctttcttccgtttgatgccaaaaatggtattgtttataatacacttaaaataattcatatccaagttgttgtataaaaaaataaacaatgtgatttcccaaaaaaaaaaaataataagtgattggcatgcaacatcagattttgttgtccaagtggtcacatttgaaatatttgagtttacatttttctcactaatatctttatattgaacttcaatctgtctagctataatcaaatggttgactaaaatggttaataagcttcaattaaattcaaattatttgaaagaacttgaatttaaatcttaacttgaacataaccccgaagtggatgatttacccaaaaaaattacctaactactctaataatcattttaacaataagagtattttactcaaagaaatttatataatcacaaaatcctagtatactcaatgattttcttaaaaaactattataaccagaaatgacaatatgagacaaatataattattttaataaaaatatctaaattagaattatttgaaaatgtaaaactatattattttttgtagttttccaaactttattaagcatcgtttaattgcacacaaaatttactccaattttttttagtctctgaaagttgctttttggtccatgttttagttttggtaattataattaagtttgttcatattaattagttctaatgtgtgtactatatagatcatactaattttgttcccaaactcatcttagttaggatctttgtatctactataagattaatccttgtgccgatataagaatatttgtcatgttatcaactatcacaatattgtattggtgagatcatataagatttgatgtcaacatcttcgtaaaggtgtcagattcgattctccccggtatcaatttaagtgagctaatttagcttcttaaaaaataaaatcaaacaacttttacataaactcagtgaaaacttggatataaagtatccttatactactctttagtcttgattagtctctgcaaagatatttatatgtactttgtattatcataagaacattcattgacattttaagttaatgaattactaacatgtcaactcttattctagccaacagttactttgttccctccacattctctttgaaatagtcaaacgtatccaatcatgcatgtctgttctgatcataacagcaaaagcatgtgtatagaaaattgatagttgaattagagtcattttccataaaaaaatattcaataagtgtgacattatttttcgtatgaattaatccattttttgctgatttgagattctttctttctttgcttcttgctttccttcatcagccattttttttgttttctctttctctctctcttcttgattca

SEQ ID NO: 14 - protein, 83 aa, Lotus japonicus
VVEVALQCLEPFSTYRPSMVAIVRELEDALIIENNASEYMKSIDSLGGSNRYSIVIEKRVLPSTTSTAESTITTQSLSHPQPR

SEQ ID NO: 15 - protein, 83 aa, synthetic construct
VVEVALQCLEPFSTYRPSMVAIVRELEDALIIENNAAEYMKSIDALGGANRYAIVIEKRVLPSTTSTAESTITTQSLSHPQPR

SEQ ID NO: 16 - protein, 83 aa, synthetic construct
VVEVALQCLEPFSTYRPSMVAIVRELEDALIIENNADEYMKSIDDLGGDNRYDIVIEKRVLPSTTSTAESTITTQSLSHPQPR

SEQ ID NO: 17 - protein, 83 aa, synthetic construct
VVEVALQCLEPFSTYRPSMVAIVRELEDALIIENNASEYMKSIDSLGGSNRYSIVIEKRVLPAAAAAAEAAITTQALAHPQPR

SEQ ID NO: 18 - protein, 83 aa, synthetic construct
VVEVALQCLEPFSTYRPSMVAIVRELEDALIIENNAAEYMKSIDALGGANRYAIVIEKRVLPAAAAAAEAAITTQALAHPQPR

SEQ ID NO: 19 - protein, 83 aa, synthetic construct
VVEVALQCLEPFSTYRPSMVAIVRELEDALIIENNADEYMKSIDDLGGDNRYDIVIEKRVLPDADDDAEDDITTQDLDHPQPR

SEQ ID NO: 20 - protein, 62 aa, synthetic construct
VVEVALQCLEPFSTYRPSMVAIVRELEDALIIENNASEYMKSIDSLGGSNRYSIVIEKRVLP

SEQ ID NO: 21 - protein, 36 aa, synthetic construct
VVEVALQCLEPFSTYRPSMVAIVRELEDALIIENNA

SEQ ID NO: 22 - protein, 69 aa, Lotus japonicus
YRPSMVAIVRELEDALIIENNASEYMKSIDSLGGSNRYSIVIEKRVLPSTTSTAESTITTQSLSHPQPR

SEQ ID NO: 23 - protein, 69 aa, Medicago truncatula
YRPCMVDIVRELEDALIIENNASEYMKSIDSLGGSNRYSIVMDKRALPSTTSTAESTITTQTLSHPQPR

SEQ ID NO: 24 - protein, 76 aa, Manihot esculenta
YRPCMADIVRELEDALIIENNASEYMKSIDSIGGYSLGGSNRFSIVIDKKNVIPPTPTPTEPSPINAQAMAPPEPR

SEQ ID NO: 25 - protein, 68 aa, Solanum lycopersicum
YRPCMADIVRELEDALIIENNASEYLKSLDSFGGSNRFSVERSIVLPPIKSQTEPSSLLSKPAPPQPR

SEQ ID NO: 26 - protein, 73 aa, Oryza sativa
FRPSMEDVVRELEDALIIENNASEYMRSIESTGTLGSNRYLSIDRKMFASGSARFASFDATKGHLQTMPSLPG

SEQ ID NO: 27 - protein, 70 aa, Zea mays
FRPTMEDVLRELEDALIIENNASEYMRSIESTGTLGSNRYLSIDRKMFASGSARIESMKGNLQAMPSLPR

SEQ ID NO: 28 - protein, 82 aa, Hordeum vulgare
FRPSMEDIARELEDALIIENNASEYMRSMESTGTFGSNRYLSIDRKMFASGSARMDPAKFASGSGRIDAGKGPLQAMPSLPR

SEQ ID NO: 29 - protein, 65 aa, Arabidopsis thaliana
NRPRMQEVIVAIQDAIRIERGNENGLKSSSSSSSKAQSSRKTLLTSFLELESPDISRNSLAPAAR

SEQ ID NO: 30 - protein, 70 aa, synthetic construct
YRPXMEDIVRELEDALIIENNASEYMKSIDSGGGSNRYSXXDRKMFAXPSXRTXTAXSXIXSQXMPPXPR

SEQ ID NO: 31 - protein, 60 aa, Lotus japonicus
FPGRSVIHRDIKSSNILLDHSMCAKVADFGFSKYAPQEGDSYVSLEVRGTAGYLDPEYYK

SEQ ID NO: 32 - protein, 60 aa, synthetic construct
FPGRSVIHRDIKSANILLDHAMCAKVADFGFAKYAPQEGDAYVALEVRGTAGYLDPEYYK

SEQ ID NO: 33 - protein, 60 aa, synthetic construct
FPGRSVIHRDIKSDNILLDHDMCAKVADFGFDKYAPQEGDDYVDLEVRGTAGYLDPEYYK

SEQ ID NO: 34 - protein, 60 aa, synthetic construct
FPGRSVIHRDIKSXNILLDHXMCAKVADFGFXKYAPQEGDXYVXLEVRGTAGYLDPEYYK